(19) United States
(12) Patent Application Publication
(10) Pub. No.: US 2024/0420797 A1
(43) Pub. Date: Dec. 19, 2024

(54) METHODS AND SYSTEMS FOR DETECTING COPY NUMBER ALTERATIONS

(71) Applicant: Foundation Medicine, Inc., Boston, MA (US)

(72) Inventors: Jason D. HUGHES, Providence, RI (US); Bernard FENDLER, Auburndale, MA (US); Justin NEWBERG, Cambridge, MA (US)

(73) Assignee: Foundation Medicine, Inc., Boston, MA (US)

(21) Appl. No.: 18/697,958

(22) PCT Filed: Oct. 7, 2022
(86) PCT No.: PCT/US2022/077781

§ 371 Date: Apr. 2, 2024

Related U.S. Application Data
(60) Provisional application No. 63/253,972, filed on Oct. 8, 2021.

(51) Int. Cl. G16B 20/10; C12Q 1/6806; C12Q 1/6855; C12Q 1/6874; G16B 45/00; G16H 20/10; G16H 50/20; G16H 50/30

(52) U.S. Cl. CPC G16B 20/10; C12Q 1/6806; C12Q 1/6855; C12Q 1/6874; G16B 45/00; G16H 20/10; G16H 50/20; G16H 50/30

(57) ABSTRACT

Methods and systems for calling copy number alterations (CNAs), including methods and systems for fitting a grid-based copy number model to sequence read data are described herein. The method can include generating a minor allele coverage ratio and a major allele coverage ratio for a plurality of genetic loci, transforming the allele coverage data, and fitting a copy number grid model to the data. The fit copy number grid model may then be used to assign a copy number state or call a copy number alteration. The copy number grid model and the transformed allele coverage ratio data may be displayed for consideration of the presented model, which allows for more efficient data interpretation and calling of a copy number state or copy number alteration.

### CROSS-REFERENCE TO RELATED APPLICATIONS

This application claims the benefit of U.S. Provisional Application No. 63/253,972 filed Oct. 8, 2021, which is hereby incorporated by reference in its entirety.

### FIELD OF THE INVENTION

The present disclosure relates generally to methods and systems for analyzing genomic profiling data, and more specifically to methods and systems for calling of copy number alterations using genomic profiling data.

### BACKGROUND

Structural variants (SVs) are large genomic alterations that typically comprise alterations of at least 50 base pairs (bp) in length (Mahmoud, et al. (2019), “Structural variant calling: the long and the short of it”, Genome Biology 20:246). These large genomic alterations can be classified as deletions, duplications, insertions, inversions, and translocations, and describe different combinations of DNA gains, losses, or rearrangements.

Abnormal chromosomal imbalance, commonly referred to as aneuploidy, often occurs during cell reproduction in which DNA repair mechanisms inadequately monitor and correct mistakes. While aneuploidy can be indicative of disease in germline cells, in somatic cells Copy Number Alterations (CNAs) often have been associated with tumorigenesis and can identify key genetic drivers, leading to increased population pressure towards the genetic changes that promote growth. As such, metastatic cancers generally show increased aneuploidy affecting genetic pathways which either correct copying mistakes or genes that promote cell growth.

Typical representations of data associated with copy-number analysis, namely the allele frequency or coverage ratio, are generally sorted genomically, with a ratio transformation to log₂(R). Often the allele frequency plot is folded such that only the minor allele frequencies are shown. Since coverage is conserved, the minor and major allele frequencies are redundant and no information is lost in this representation. After a model has been fit, it is then customary to overlay those copy-number states on top of the predicted ratios and allele frequencies. Coverage ratio plots generally show directionality associated with copy-number events. If an amplification occurs, the coverage ratio increases. If a deletion occurs, the coverage ratio decreases. It is more challenging to decipher amplifications and deletions from the allele frequency plots. While these representations are intuitive since targets are sorted genomically and the observables are plotted as a function of this sorting, it can often be challenging to visually identify copy-number states and how well the states correspond to both the allele frequency and coverage ratio.

### SUMMARY OF THE INVENTION

Described herein are methods and systems for calling copy number alterations (CNAs), including methods and systems for fitting a grid-based copy number model (i.e., a “copy number grid model”) to sequence read data. Advantageously, fitting the copy number grid model to sequence read data allows for display of the data that makes it more efficient to interpret the data and call a copy number state or copy number alteration, which is often difficult to accurately call. That is, the copy number grid model provides a visualization of data associated with both coverage ratio and allele frequency, and their associated errors, on the same graph, while simultaneously overlaying the predicted copy number state for a more complete presentation. Display of the of the resulting overlay facilitates manual calling a copy number for a genomic segment or genomic locus, or manual confirmation a call (for example, a call made by an automated process) for a copy number for a genomic segment or genomic locus.

In some implementations, the method for calling copy number alterations (CNAs) comprises: providing a plurality of nucleic acid molecules obtained from a sample from a subject; ligating one or more adapters onto one or more nucleic acid molecules from the plurality of nucleic acid molecules; amplifying the one or more ligated nucleic acid molecules from the plurality of nucleic acid molecules; capturing amplified nucleic acid molecules from the amplified nucleic acid molecules; sequencing, by a sequencer, the captured nucleic acid molecules to obtain a plurality of sequence reads that represent the captured nucleic acid molecules, thereby generating sequence read data for a genome of the sample; receiving, at one or more processors, the sequence read data; generating for a plurality of genetic loci, using the one or more processors, a minor allele coverage ratio and a major allele coverage ratio; segmenting, using the one or more processors, the genome into a plurality of genomic segments; generating, using the one or more processors, for genetic loci in the plurality of genetic loci, copy number grid model input data comprising (i) a difference between the major allele coverage ratio and the minor allele coverage ratio, and (ii) a sum of the major allele coverage ratio and the minor allele coverage ratio; fitting, using the one or more processors, a plurality of copy number grid models comprising allowed copy number states to the copy number grid model input data; selecting, using the one or more processors, a copy number grid model from the plurality of copy number grid models; and assigning, using the one or more processors, a copy number state for at least a portion of the plurality of genomic segments based on the selected copy number grid model.

In some implementations, the one or more adapters comprise amplification primers, flow cell adaptor sequences, substrate adapter sequences, or sample index sequences.

In some implementations, the captured nucleic acid molecules are captured from the amplified nucleic acid molecules by hybridization to one or more bait molecules. In some implementations, the one or more bait molecules comprise one or more nucleic acid molecules, each comprising a region that is complementary to a region of a captured nucleic acid molecule.

In some implementations, amplifying nucleic acid molecules comprises performing a polymerase chain reaction (PCR) amplification technique, a non-PCR amplification technique, or an isothermal amplification technique.

In some implementations, the sequencing comprises use of a massively parallel sequencing (MPS) technique, whole genome sequencing (WGS), whole exome sequencing, targeted sequencing, direct sequencing, or Sanger sequencing technique. For example, in some implementations, the sequencing comprises massively parallel sequencing, and the massively parallel sequencing technique comprises next generation sequencing (NGS). In some implementations, the sequencer comprises a next generation sequencer.

In some implementations, the method for calling copy number alterations (CNAs) comprises: receiving, at one or more processors, sequence read data for a plurality of sequence reads associated with a plurality of nucleic acid molecules obtained from a sample from a subject; generating for a plurality of genetic loci, using the one or more processors, a minor allele coverage ratio and a major allele coverage ratio; segmenting, using the one or more processors, the genome into a plurality of genomic segments; generating, using the one or more processors, for genetic loci in the plurality of genetic loci, copy number grid model input data comprising (i) a difference between the major allele coverage ratio and the minor allele coverage ratio, and (ii) a sum of the major allele coverage ratio and the minor allele coverage ratio; fitting, using the one or more processors, a plurality of copy number grid models comprising allowed copy number states to the copy number grid model input data; selecting, using the one or more processors, a copy number grid model from the plurality of copy number grid models; and assigning, using the one or more processors, a copy number state for at least a portion of the plurality of genomic segments based on the selected copy number grid model

In some implementations of any of the above methods, the segmenting is based on the minor allele coverage ratio, the major allele coverage ratio, or a total coverage ratio.

In some implementations of any of the above methods, selecting the copy number grid model from the plurality of copy number grid models comprises: determining, for each genomic segment, a distribution of the copy number grid model input data; identifying, for each genomic segment, a distance between the distribution and a closest copy number state; and determining an overall model fit score based on an average distance across the plurality of genomic segments.

In some implementations of any of the above methods, different copy number grid models in the plurality of copy number grid models are initialized using different initial tumor purity estimates and tumor ploidy estimates.

In some implementations of any of the above methods, fitting the plurality of copy number grid models to the copy number grid model input data comprises, for each copy number grid model: fitting the allowed copy numbers states of the copy number grid model to the copy number grid model input data based on an initial tumor purity estimate and an initial tumor ploidy estimates; and iteratively: assigning a preliminary copy number to each genomic segment in the plurality of genomic segments, determining an updated tumor ploidy estimate and updated tumor purity estimate based on the preliminary copy number assignments, and re-fitting the allowed copy numbers states to the copy number grid model input data based on the updated tumor ploidy estimate and updated tumor purity estimate.

In some implementations of any of the above methods, the initial tumor purity is bound by a preselected tumor purity lower limit and a preselected tumor purity upper limit. In some implementations, the preselected tumor purity lower limit is 0 and the preselected tumor purity upper limit is 1.

In some implementations of any of the above methods, the initial tumor ploidy is bound by a preselected tumor ploidy lower limit. In some implementations, the preselected tumor ploidy lower limit is about 1.1 to about 1.5. In some implementations, the initial tumor ploidy is bound by a preselected tumor ploidy upper limit. In some implementations, the preselected tumor ploidy lower upper is about 6 to about 10.

In some implementations of any of the above methods, the assigned copy number state for each of the plurality of genomic segments is a total copy number count for the genomic segment, a minor allele copy number count for the genomic segment, or a major allele copy number count for the genomic segment.

In some implementations of any of the above methods, the segmenting step is performed using a circular binary segmentation (CBS) method, a maximum likelihood method, a hidden Markov chain method, a walking Markov method, a Bayesian method, a long-range correlation method, or a change point method. In some implementations, the segmenting step is performed using a change point method, and the change point method is a pruned exact linear time (PELT) method.

In some implementations of any of the above methods, the method further comprises overlaying the selected copy number grid model and the copy number grid model input data to generate an overlay. The overlay may then be displayed. In some implementations, the overlay is displayed using an electronic display.

In some implementations of any of the above methods, the method further comprises calling a copy number alteration for the one or more genetic loci or one or more genomic segments based on one or more assigned copy number states or a sum coverage ratio threshold. In some implementations, the called copy number alteration for the one or more genetic loci or the one or more genomic segments is used to diagnose or confirm a diagnosis of disease in the subject.

In some implementations of any of the above methods, the method further comprises generating a genomic profile for the subject comprising the called copy number alteration for the one or more genetic loci or the one or more genomic segments. In some implementations, the genomic profile for the subject further comprises results from a comprehensive genomic profiling test, a gene expression profiling test, a cancer hotspot panel test, a DNA methylation test, a DNA fragmentation test, an RNA fragmentation test, or any combination thereof. In some implementations, the genomic profile for the subject further comprises results from a nucleic acid sequencing-based test. In some implementations of any of the above methods, the method further comprises selecting an anticancer agent, administering an anticancer agent, or applying an anticancer therapy to the subject based on the generated genomic profile.

In some implementations of any of the above methods, the method further comprises the called copy number alteration for the one or more genetic loci or the one or more genomic segments is used in making a suggested treatment decision for the subject.

In some implementations of any of the above methods, the method further comprises the called copy number alteration for the one or more genetic loci or the one or more genomic segments is used in applying or administering a treatment to the subject.

Also described herein is a method for diagnosing a disease, the method comprising determining that a subject has the disease based on a called copy number alteration for the one or more genetic loci or the one or more genomic segments, wherein the called copy number alteration for the one or more genetic loci or the one or more genomic segments is determined according to any one of the above methods.

Also described herein is a method of identifying a subject as being eligible for a clinical trial for a treatment of a disease, comprising: determining that the subject has the disease the disease based on a called copy number alteration for the one or more genetic loci or the one or more genomic segments, wherein the called copy number alteration for the one or more genetic loci or the one or more genomic segments is determined according to any one of the above methods. In some implementations, the method further comprises enrolling the subject in the clinical trial. In some implementations, the method further comprises administering the treatment to the subject. In some implementations, the treatment is an anticancer therapy.

In some implementations, the disease is cancer. In some implementations, the disease is a genetic disorder, for example a disease associated with a chromosomal aneuploidy (e.g., Down syndrome, Edwards syndrome, or Patau syndrome) or Fragile X. In some implementations, disease is cancer, and the method further comprises selecting an anticancer therapy to administer to the subject based on the called copy number alteration for the one or more genetic loci or the one or more genomic segments.

Also described herein is a method of selecting an anticancer therapy for a subject having cancer, the method comprising: responsive to a copy number alteration for the one or more genetic loci or the one or more genomic segments called according to the above methods, selecting an anticancer therapy for the subject. In some implementations, the method further comprises determining an effective amount of an anticancer therapy to administer to the subject based on the called copy number alteration for the one or more genetic loci or the one or more genomic segments. In some implementations, the method further comprises administering the anticancer therapy to the subject based on the called copy number alteration for the one or more genetic loci or the one or more genomic segments.

Also described herein is method of treating a cancer in a subject, comprising: responsive to a called copy number alteration for the one or more genetic loci or the one or more genomic segments called according to the above method, administering an effective amount of an anticancer therapy to the subject.

Also described herein is a method for monitoring tumor progression or recurrence in a subject, the method comprising: calling a copy number alteration for one or more genetic loci or one or more genomic segments using a first sample obtained from the subject at a first time point according to the above method; calling a copy number alteration for the one or more genetic loci or one or more genomic segments using a second sample obtained from the subject at a second time point; and comparing the first called a copy number alteration to the second called a copy number alteration for the one or more genetic loci or the one or more genomic segments, thereby monitoring the cancer progression or recurrence. In some implementations, the called a copy number alteration for the one or more genetic loci or one or more genomic segments in using the second sample is determined according to the above method. In some implementations, the method further comprises adjusting an anticancer therapy in response to the tumor progression. In some implementations, the method further comprises adjusting a dosage of the anticancer therapy or selecting a different anticancer therapy in response to tumor progression. In some implementations, the method further comprises administering the adjusted anticancer therapy to the subject. In some implementations, the first time point is before the subject has been administered an anticancer therapy, and the second time point is after the subject has been administered the anticancer therapy.

In some implementations of the above methods, the subject has a cancer, is at risk of having a cancer, is being routine tested for cancer, or is suspected of having a cancer.

In some implementations of the above methods, the anticancer therapy or anticancer therapy comprises chemotherapy, radiation therapy, immunotherapy, a targeted therapy, or surgery.

In some implementations of the above methods, the cancer or tumor is a solid cancer. In some implementations of the above methods, cancer or tumor is a hematological cancer. In some implementations of the above methods, the cancer or tumor is a B cell cancer (multiple myeloma), a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.

In some implementations of the above methods, the method further comprises generating, by the one or more processors, a report indicating the copy number state or called copy number alteration for one or more genomic loci or one or more genomic segments. In some implementations of the above methods, the method further comprises transmitting the report to the subject or a healthcare provider. In some implementations, the report is transmitted via a computer network or a peer-to-peer connection.

In some implementations of the above methods, the subject is suspected of having or is determined to have cancer.

In some implementations of the above methods, the method further comprises obtaining the sample from the subject.

In some implementations of the above methods, the sample comprises a tissue biopsy sample, a liquid biopsy sample, or a normal control.

In some implementations of the above methods, the sample is a liquid biopsy sample and comprises blood, plasma, cerebrospinal fluid, sputum, stool, urine, or saliva.

In some implementations of the above methods, the sample is a liquid biopsy sample and comprises circulating tumor cells (CTCs).

In some implementations of the above methods, the sample is a liquid biopsy sample and comprises cell-free DNA (cfDNA), circulating tumor DNA (ctDNA), or any combination thereof.

In some implementations of the above methods, the plurality of nucleic acid molecules comprises a mixture of tumor nucleic acid molecules and non-tumor nucleic acid molecules. In some implementations, the tumor nucleic acid molecules are derived from a tumor portion of a heterogeneous tissue biopsy sample, and the non-tumor nucleic acid molecules are derived from a normal portion of the heterogeneous tissue biopsy sample. In some implementations, the sample comprises a liquid biopsy sample, and the tumor nucleic acid molecules are derived from a circulating tumor DNA (ctDNA) fraction of the liquid biopsy sample, and the non-tumor nucleic acid molecules are derived from a non-tumor, cell-free DNA (cfDNA) fraction of the liquid biopsy sample.

Also described herein is a system comprising: one or more processors; and a memory communicatively coupled to the one or more processors and configured to store instructions that, when executed by the one or more processors, cause the system to: receive, at the one or more processors, sequence read data for a plurality of sequence reads associated with a plurality of nucleic acid molecules obtained from a sample from a subject; generate for a plurality of loci, using the one or more processors, a minor allele coverage ratio and a major allele coverage ratio; segment, using the one or more processors, a genome into a plurality of genomic segments; generate, using the one or more processors, for loci in the plurality of loci, copy number grid model input data comprising (i) a difference between the major allele coverage ratio and the minor allele coverage ratio, and (ii) a sum of the major allele coverage ratio and the minor allele coverage ratio; fit, using the one or more processors, a plurality of copy number grid models comprising allowed copy number states to the copy number grid model input data; select, using the one or more processors, a copy number grid model from the plurality of copy number grid models; and assign, using the one or more processors, a copy number state for at least a portion of the plurality of genomic segments based on the selected copy number grid model.

In some implementations of the system, the genome is segmented based on the minor allele coverage ratio, the major allele coverage ratio, or a total coverage ratio.

In some implementations of the system, the instructions that cause the system to select the selected copy number grid model comprise instructions that cause the system to: determine, for each genomic segment, a distribution of the copy number grid model input data; identify, for each genomic segment, a distance between the distribution and a closest copy number state; and determine an overall model fit score based on an average distance across the plurality of genomic segments.

In some implementations of the system, different copy number grid models in the plurality of copy number grid models are initialized using different initial tumor purity estimates and tumor ploidy estimates.

In some implementations of the system, the instructions that fit allowed copy number states to the transformed coverage ratio data comprise instructions that cause the system to: fit the allowed copy numbers states to the copy number grid model input data based on the initializing tumor purity estimate and the initial tumor ploidy estimate; and iteratively: assign a preliminary copy number to each segment in the plurality of segments, determine an updated tumor ploidy estimate and updated tumor purity estimate based on the preliminary copy number assignments, and re-fit the allowed copy numbers states to the copy number grid model input data based on the updated tumor ploidy estimate and updated tumor purity estimate.

In some implementations of the system, the initial tumor purity is bound by a preselected tumor purity lower limit and a preselected tumor purity upper limit. In some implementations, the preselected tumor purity lower limit is 0 and the preselected tumor purity upper limit is 1.

In some implementations of the system, the initial tumor ploidy is bound by a preselected tumor ploidy lower limit. In some implementations, the preselected tumor ploidy lower limit is about 1.1 to about 1.5. In some implementations of the system, the initial tumor ploidy is bound by a preselected tumor ploidy upper limit. In some implementations of the system, the preselected tumor ploidy lower upper is about 6 to about 10.

In some implementations of the system, the assigned copy number state for each of the plurality of genomic segments is a total copy number count for the genomic segment, a minor allele copy number count for the genomic segment, or a major allele copy number count for the genomic segment.

In some implementations of the system, the genome is segmented into a plurality of genomic segments using a circular binary segmentation (CBS) method, a maximum likelihood method, a hidden Markov chain method, a walking Markov method, a Bayesian method, a long-range correlation method, or a change point method. In some implementations, the segmenting step is performed using a change point method, and the change point method is a pruned exact linear time (PELT) method.

In some implementations of the system, the system further comprises an electronic display, and the instructions further comprise instructions that, when executed by the one or more processors, cause the system to overlay the selected copy number grid model and the copy number grid model input data to generate an overlay, and display the overlay on the electronic display.

In some implementations of the system, the system further comprises instructions that, when executed by the one or more processors, cause the system to call a copy number alteration for the one or more genetic loci or one or more genomic segments based on one or more assigned copy number states or a sum coverage ratio threshold.

In some implementations of the system, the system further comprises instructions that, when executed by the one or more processors, cause the system to generate a report indicating the copy number state or called copy number alteration for one or more genomic loci or one or more genomic segments. In some implementations, the system further comprises instructions that, when executed by the one or more processors, cause the system to transmit the report to the subject or a healthcare provider. In some implementations, the report is transmitted via a computer network or a peer-to-peer connection.

Also described herein is a non-transitory computer-readable storage medium storing one or more programs, the one or more programs comprising instructions, which when executed by one or more processors of a system, cause the system to: receive, at the one or more processors, sequence read data for a plurality of sequence reads associated with a plurality of nucleic acid molecules obtained from a sample from a subject; generate for a plurality of loci, using the one or more processors, a minor allele coverage ratio and a major allele coverage ratio; segment, using the one or more processors, a genome into a plurality of genomic segments; generate, using the one or more processors, for loci in the plurality of loci, copy number grid model input data comprising (i) a difference between the major allele coverage ratio and the minor allele coverage ratio, and (ii) a sum of the major allele coverage ratio and the minor allele coverage ratio; fit, using the one or more processors, a plurality of copy number grid models comprising allowed copy number states to the copy number grid model input data; select, using the one or more processors, a selected copy number grid model from the plurality of copy number grid models; and assign, using the one or more processors, a copy number state for at least a portion of the plurality of genomic segments based on the selected copy number grid model.

In some implementations of the non-transitory, computer-readable storage medium, the genome is segmented based on the minor allele coverage ratio, the major allele coverage ratio, or a total coverage ratio.

In some implementations of the non-transitory computer-readable storage medium, the instructions that cause the system to select the selected copy number grid model comprise instructions that cause the system to: determine, for each genomic segment, a distribution of the copy number grid model input data; identify, for each genomic segment, a distance between the distribution and a closest copy number state; and determine an overall model fit score based on an average distance across the plurality of genomic segments.

In some implementations of the non-transitory computer-readable storage medium, different copy number grid models in the plurality of copy number grid models are initialized using different initial tumor purity estimates and tumor ploidy estimates.

In some implementations of the non-transitory computer-readable storage medium, the instructions that cause the system to fit allowed copy number states to the copy number grid model input data comprise instructions that cause the system to: fit the allowed copy numbers states to the copy number grid model input data based on the initializing tumor purity estimate and the initial tumor ploidy estimate; and iteratively: assign a preliminary copy number to each segment in the plurality of segments; determine an updated tumor ploidy estimate and updated tumor purity estimate based on the preliminary copy number assignments; and re-fit the allowed copy numbers states to the copy number grid model input data based on the updated tumor ploidy estimate and updated tumor purity estimate.

In some implementations of the non-transitory computer-readable storage medium, the initial tumor purity is bound by a preselected tumor purity lower limit and a preselected tumor purity upper limit. In some implementations, the preselected tumor purity lower limit is 0 and the preselected tumor purity upper limit is 1.

In some implementations of the non-transitory computer-readable storage medium, the initial tumor ploidy is bound by a preselected tumor ploidy lower limit. In some implementations, the preselected tumor ploidy lower limit is about 1.1 to about 1.5. In some implementations, the initial tumor ploidy is bound by a preselected tumor ploidy upper limit. In some implementations, the preselected tumor ploidy lower upper is about 6 to about 10.

In some implementations of the non-transitory computer-readable storage medium, the assigned copy number state is a total copy number count for the genomic segment, a minor allele copy number count for the genomic segment, or a major allele copy number count for the genomic segment.

In some implementations of the non-transitory computer-readable storage medium, the genome is segmented into a plurality of genomic segments using a circular binary segmentation (CBS) method, a maximum likelihood method, a hidden Markov chain method, a walking Markov method, a Bayesian method, a long-range correlation method, or a change point method. In some implementations, the segmenting step is performed using a change point method, and the change point method is a pruned exact linear time (PELT) method.

In some implementations of the non-transitory computer-readable storage medium, the system comprises an electronic display, and wherein the instructions further comprise instructions that, when executed by the one or more processors, cause the system to overlay the selected copy number grid model and the copy number grid model input data to generate an overlay, and display the overlay on the electronic display.

In some implementations of the non-transitory computer-readable storage medium, the storage medium further comprises instructions that, when executed by the one or more processors, cause the system to call a copy number alteration for the one or more genetic loci or one or more genomic segments based on one or more assigned copy number states or a sum coverage ratio threshold.

In some implementations of the non-transitory computer-readable storage medium, the storage medium further comprises that, when executed by the one or more processors, cause the system to generate a report indicating the copy number state or called copy number alteration for one or more genomic loci or one or more genomic segments. In some implementations, the storage medium further comprises instructions that, when executed by the one or more processors, cause the system to transmit the report to the subject or a healthcare provider. In some implementations, the report is transmitted via a computer network or a peer-to-peer connection.

### BRIEF DESCRIPTION OF THE DRAWINGS

Various aspects of the disclosed methods, devices, and systems are set forth with particularity in the appended claims. A better understanding of the features and advantages of the disclosed methods, devices, and systems will be obtained by reference to the following detailed description of illustrative embodiments and the accompanying drawings, of which:

FIG. 1 provides an exemplary set of copy number grid points for a plot of the difference between the major allele coverage ratio and the minor allele coverage ratio against the sum of the major allele coverage ratio and the minor allele coverage ratio.

FIG. 2A shows an exemplary copy number grid in copy number space, wherein the minor allele copy number and the major allele copy number are plotted against each other.

FIG. 2B shows the exemplary copy number grid of FIG. 2A scaled by assuming a purity of 0.95 and a ploidy of 2 in the illustrated example. FIG. 2C shows the scaled copy number grid of FIG. 2B rotated by

$$θ = - \frac{π}{2} .$$

FIG. 2D shows the rotated and scaled copy number grid of FIG. 2D transformed by a translation parameter based on purity and ploidy.

FIG. 3 shows an exemplary interface that includes a selected copy number grid model with the transformed coverage ratio data, according to some embodiments.

FIG. 4 shows an exemplary method for determining a copy number state for one or more genetic loci or one or more genomic segments, according to some embodiments.

FIG. 5 depicts an exemplary computing device, in accordance with some instances of the systems described herein.

FIG. 6 depicts an exemplary computer system or computer network, in accordance with some instances of the systems described herein.

### DETAILED DESCRIPTION OF THE INVENTION

Described herein are methods and systems for calling copy number alterations (CNAs), including methods and systems for fitting a grid-based copy number model (i.e., a “copy number grid model”) to sequence read data. The copy number alterations may be called, for example, for a tumor in a subject. Samples from a subject can include, for example, a mixture of tumor and non-tumor nucleic acid molecules. While the copy number state for a healthy (e.g., non-tumor) tissue includes a single maternal allele and a single paternal allele euploid subjects, copy number alteration events in tumors make calling the copy number state of a tumor more challenging, particularly when the tumor ploidy and/or tumor purity of the sample are unknown apriori. The methods described herein allow for calling the copy number state of genomic segments in the genome of a diseased tissue (e.g., a tumor).

The methods described herein may use sequence read data to generate, for a plurality of genetic loci, a minor allele coverage ratio and a major allele coverage ratio. The genome can be segmented into a plurality of genomic segments, for example based on the minor allele coverage ratio, the major allele coverage ratio, or a total coverage ratio. The minor allele coverage ratios and the major allele coverage ratios can be transformed to generate copy number grid model input data, which can include (i) a difference between the major allele coverage ratio and the minor allele coverage ratio, and (ii) a sum of the major allele coverage ratio and the minor allele coverage ratio.

Because copy number alteration events are necessarily integer events, plotting the difference between the allele coverage ratios against the sum of the allele coverage ratios should, absent any noise in the system, provide evenly spaced grid points. In reality, the sequence read data includes noise that generally prevents a perfect match between the transformed coverage ratio data (i.e., the copy number grid model input data) and the gird points. By fitting a plurality of copy number grid models to the copy number grid model input data, a copy number grid model may be selected (for example, a best-fit copy number grid model), which can be used to identify a copy number state for at least a portion of the genomic segments. The copy number grid models include allowed copy number states. As further described herein, the copy number models in the plurality of copy number grid models may be initialized using different combinations of initial tumor purity estimates and tumor ploidy estimates, which need not be known apriori. The selected number grid model may be overlaid with the copy number grid model input data and the overlay displayed, for example on an interface output on an electronic display or a printed report. This allows for easy viewing of data, and facilitates manual calling of copy number alterations or confirmation of called copy number alterations (for example, copy number alterations called by an automated process).

The copy number state assigned using the model may be a total copy number count for a genomic segment, a minor allele copy number count for the genomic segment, or a major allele copy number count for the genomic segment. Segmentation may be based on an approximation of equal copy numbers of genomic loci within the segment. Thus, a genomic locus within a segment can be assumed to have the same copy number stat as the genomic segment itself. Copy number alterations for the one or more genetic loci can be called (e.g., a call of whether a copy number alteration has occurred, a call of a copy number increase, a call of a copy number increase, or a call of a number of calls) based on the copy number state assigned for the corresponding genomic segment.

### Definitions

Unless otherwise defined, all of the technical terms used herein have the same meaning as commonly understood by one of ordinary skill in the art in the field to which this disclosure belongs.

As used in this specification and the appended claims, the singular forms “a”, “an”, and “the” include plural references unless the context clearly dictates otherwise. Any reference to “or” herein is intended to encompass “and/or” unless otherwise stated.

As used herein, the terms “comprising” (and any form or variant of comprising, such as “comprise” and “comprises”), “having” (and any form or variant of having, such as “have” and “has”), “including” (and any form or variant of including, such as “includes” and “include”), or “containing” (and any form or variant of containing, such as “contains” and “contain”), are inclusive or open-ended and do not exclude additional, un-recited additives, components, integers, elements, or method steps.

As used herein, the term “about” a number or value refers to that number or value plus or minus 10% of that number or value. The term “about: when used in the context of a range refers to that range minus 10% of its lowest value and plus 10% of its greatest value.

As used herein, the term “subgenomic interval” (or “subgenomic sequence interval”) refers to a portion of a genomic sequence.

As used herein, the term “subject interval” refers to a subgenomic interval or an expressed subgenomic interval (e.g., the transcribed sequence of a subgenomic interval).

As used herein, the terms “variant sequence” or “variant” are used interchangeably and refer to a modified nucleic acid sequence relative to a corresponding “normal” or “wild-type” sequence. In some instances, a variant sequence may be a “short variant sequence” (or “short variant”), i.e., a variant sequence of less than about 50 base pairs in length.

The terms “allele frequency” and “allele fraction” are used interchangeably herein and refer to the fraction of sequence reads corresponding to a particular allele relative to the total number of sequence reads for a genomic locus.

The terms “variant allele frequency” and “variant allele fraction” are used interchangeably herein and refer to the fraction of sequence reads corresponding to a particular variant allele relative to the total number of sequence reads for a genomic locus.

As used herein, the term “segmentation” (or “sequence segmentation”) refers to a process for partitioning of the sequence read data into a number of non-overlapping segments that cover all sequence read data points, such that each segment of the plurality of segments is as homogeneous as possible and all sequence reads associated with a given segment have the same copy number. In some instances, segmentation may be performed by processing aligned sequence read data (or other sequencing-related data, e.g., coverage data, allele frequency data, etc., derived from the sequence read data) using any of a variety of methods known to those of skill in the art (see., e.g., Braun and Miller (1998), “Statistical methods for DNA sequence segmentation”, Statistical Science 13(2):142-162). Examples of segmentation methods include, but are not limited to, circular binary segmentation (CBS) methods, maximum likelihood methods, hidden Markov chain methods, walking Markov methods, Bayesian methods, long-range correlation methods, change point methods, or any combination thereof.

As used herein, the term “ploidy” refers to the average copy number for a plurality of gene loci in a tumor sample. In some instances, the “ploidy” of a tumor sample may differ from the number of complete sets of chromosomes in a cell, and hence the number of possible alleles for autosomal genes (i.e., genes located on numbered, non-sex chromosomes), due to the heterogeneity of the tumor sample (i.e., the variation in tumor sample purity).

It is understood that aspects and variations of the invention described herein include “consisting” and/or “consisting essentially of” aspects and variations.

When a range of values is provided, it is to be understood that each intervening value between the upper and lower limit of that range, and any other stated or intervening value in that states range, is encompassed within the scope of the present disclosure. Where the stated range includes upper or lower limits, ranges excluding either of those included limits are also included in the present disclosure.

Some of the analytical methods described herein include mapping sequences to a reference sequence, determining sequence information, and/or analyzing sequence information. It is well understood in the art that complementary sequences can be readily determined and/or analyzed, and that the description provided herein encompasses analytical methods performed in reference to a complementary sequence.

The section headings used herein are for organization purposes only and are not to be construed as limiting the subject matter described. The description is presented to enable one of ordinary skill in the art to make and use the invention and is provided in the context of a patent application and its requirements. Various modifications to the described embodiments will be readily apparent to those persons skilled in the art and the generic principles herein may be applied to other embodiments. Thus, the present invention is not intended to be limited to the embodiment shown but is to be accorded the widest scope consistent with the principles and features described herein.

The figures illustrate processes according to various embodiments. In the exemplary processes, some blocks are, optionally, combined, the order of some blocks is, optionally, changed, and some blocks are, optionally, omitted. In some examples, additional steps may be performed in combination with the exemplary processes. Accordingly, the operations as illustrated (and described in greater detail below) are exemplary by nature and, as such, should not be viewed as limiting.

The disclosures of all publications, patents, and patent applications referred to herein are each hereby incorporated by reference in their entireties. To the extent that any reference incorporated by reference conflicts with the instant disclosure, the instant disclosure shall control.

### Methods of Determining Copy Number

The methods described herein provide for a determination of a copy number state of one or more genomic segments of a genome from a sample of a subject. Sequencing read data associated with a plurality of nucleic acid molecules obtained from a sample from a subject can be used to generate, for a plurality of genetic loci, a minor allele coverage ratio and a major allele coverage ratio. The genome of the subject may be segmented into a plurality of genomic segments, for example based on the minor allele coverage ratio, the major allele coverage ratio, or a total coverage ratio. The minor allele coverage ratio and the major allele coverage ratio for each for the genetic loci can be transformed to generate copy number grid model input data, which can include (i) a difference between the major allele coverage ratio and the minor allele coverage ratio, and (ii) a sum of the major allele coverage ratio and the minor allele coverage ratio. A plurality of copy number grid models that include allowed copy number states can be fit to the copy number grid model input data. Different copy number grid models in the plurality of copy number grid models may be initialized using different initial tumor purity estimates and tumor ploidy estimates. A number grid model can be selected from the plurality of copy number gird models. A copy number state for at least a portion of the plurality of genomic segments can then be assigned based on the selected copy number grid model.

Major and minor allele frequencies (e.g., a SNP allele frequency) and total coverage ratio (i.e., the sum of the major and minor allele coverage, normalized by a normalization factor) may be determined from the sequence read data. The coverage ratio data for the sample (e.g., a patient tumor sample) may be determined, for example, by aligning a plurality of sequence reads that overlap one or more genetic loci within one or more subgenomic intervals in the sample and in a control (e.g., a paired normal control, a process-matched control, or a “panel of normal” control) to a reference genome (e.g., the GRCh38 human reference genome), and determining a number of sequence reads that overlap each of the one or more gene loci within the one or more subgenomic intervals in the sample and in the control in order to normalize the coverage for the tumor sample to that in the control. In some instances, e.g., if a paired normal control sample is not available, a process-matched control (e.g., a mixture of DNA from a plurality of HapMap cell lines) may be used instead of the paired normal control to normalize coverage. In some instances, e.g., if a paired normal control sample is not available, a “panel of normal” control may be used instead of the paired normal control to normalize coverage.

In some instances, a “panel of normal” or “Tangent normalization” control method may be used to normalize sequencing coverage (see, e.g., Tabak, et al. (2019) “The Tangent copy-number inference pipeline for cancer genome analyses”, https://www.biorxiv.org/content/10.1101/566505v1.full.pdf). The Tangent normalization method is a method of normalizing tumor data in order to deal with noise in the data. Specifically, the Tangent method deals with reducing systemic noise resulting from differences in the experimental conditions under which sequencing data from tumors and/or their normal controls were generated. It has been shown that the Tangent normalization method yields a greater reduction in noise than conventional normalization methods.

In an exemplary Tangent normalization method, let n_{N} be the number of normal samples and n_{T} be the number of tumor samples. Let i be an element of the set {1, 2, . . . , n_{N}} and j be an element of the set {1, 2, . . . , n_{T}}. Define Nᵢ to be the vector of log 2 copy-ratio intensities in genomic order for the i^{th} normal sample. Similarly, define Tⱼ to be the vector of log 2 copy-ratio intensities in genomic order for the j^{th} tumor sample. The normal sample vectors and the tumor sample vectors are elements of the M-dimensional vector space of all possible coverage profiles. Now define a reference subspace N of the vector space of all possible coverage profiles to be the space that contains all linear combinations of the vectors {N₁, N₂, . . . , N_{nN}} of normal samples. Nis called the “noise space” and is an (n_{N}−1)-dimensional plane.

Given this setup, the Tangent normalization method proceeds as follows. Start by determining, for each tumor sample vector Tⱼ, the vector in the noise space N that is closest to Tⱼ using a Euclidean metric. Denote this vector p(Tⱼ), the projection of Tⱼ onto N. p(Tⱼ) represents the profile of a normal sample characterized under similar conditions to Tⱼ. The normalization of Tⱼ can now be computed by calculating the difference between Tⱼ and the projection p(Tⱼ) of Tⱼ onto N:

$$Normalization of {T}_{j} = {T}_{j} - p ( {T}_{j} )$$

The projection p(Tⱼ) can be computed directly using standard linear algebra techniques.

In some instances, the allele fraction data for the sample (e.g., a patient tumor sample) is determined by aligning a plurality of sequence reads that overlap one or more gene loci within one or more subgenomic intervals in the sample to a reference genome (e.g., the GRCh38 human reference genome), detecting a number of different alleles present at the one or more gene loci in the one or more subgenomic intervals in the sample, and determining an allele fraction for the different alleles present at the one or more gene loci by dividing the number of sequence reads identified for a given allele sequence by the total number of sequence reads identified for the gene locus.

Given the major and minor allele fraction and the coverage ratio for a given genetic locus, the haploid coverage ratios (i.e., the minor allele coverage ratio and the major allele coverage ratio) can be generated from the sequence read data. The minor allele coverage ratio is proportional to the minor allele frequency and the total coverage ratio. Similarly, the major allele coverage ratio is proportional to the major allele frequency and the total coverage ratio. A scaling factor may be used to scale the minor and major allele coverage ratios to the total coverage ratios, for example by multiplying the product of the total coverage ratio and the allele frequency by 2. For example, the major allele coverage ratio (R_{A}) and the minor allele coverage ratio (R_{B}) may be determined as follows: R_{A}=2*R*F_{A} and R_{B}=2*R*F_{B}, wherein R is the total coverage ratio, and F_{A} and F_{B} are the major allele fraction and the minor allele fraction, respectively.

The genome for the sample (e.g., a patient tumor sample) may be segmented to define genomic segments. The genetic loci are accordingly binned within the genomic segments. The genome may be segmented, for example, by aligning a plurality of sequence reads that overlap one or more gene loci within one or more subgenomic intervals in the sample to a reference genome (e.g., the GRCh38 human reference genome), and processing the aligned sequence read data (or other sequencing-related data, e.g., total coverage ratio data, allele frequency data, etc., derived from the sequence read data) using a segmentation algorithm (e.g., a circular binary segmentation (CBS) method, a maximum likelihood method, a hidden Markov chain method, a walking Markov method, a Bayesian methods, a long-range correlation method, a change point method, or any combination thereof) to generate a plurality of non-overlapping segments such that the sequence associated with a given segment have the same copy number. In some instances, the segmentation data for the sample (e.g., a patient tumor sample) may be generated using a pruned exact linear time (PELT) method to determine a number of segments required to properly account for the aligned sequence read data (or other sequencing-related data, e.g., coverage ratio data, allele frequency data, etc., derived from the sequence read data), where each segment (and the sequence reads associated with the segment) has the same copy number.

The allele coverage ratio for a genomic locus is a function of the allelic copy number, the tumor purity of the sample (i.e., the proportion of nucleic acid molecules in the sample that are of tumor origin, as opposed to non-tumor origin), and tumor ploidy. That is:

$$\begin{matrix}{R}_{x} = \frac{1 + {C}_{x} g}{1 + \frac{ψ}{2} g} \\ g = \frac{ρ}{1 - ρ}\end{matrix}$$

wherein ρ is the tumor purity (also referred to as a tumor fraction) and ψ is the tumor ploidy, and x can refer to the major allele (A) or the minor allele (B). Because the tumor purity, tumor ploidy, and copy number are not directly detected (and are not known apriori), model fitting allows for an approximation of these values based on the minor allele coverage ratio and the major allele coverage ratio.

The data minor allele coverage ratio and the major allele coverage ratio can be transformed to generate transformed coverage ratio data (i.e., copy number grid model input data) so that copy number grid models can be fit to the data. The transformed coverage ratio data can include (i) a difference between the major allele coverage ratio and the minor allele coverage ratio (i.e., R_{A}−R_{B}), and (i) a sum of the major allele coverage ratio and the minor allele coverage ratio (i.e., R_{A}+R_{B}). The difference between the major allele coverage ratio and the minor allele coverage ratio is related to copy number, tumor purity, and ploidy as follows:

$${R}_{A} - {R}_{B} = \frac{( {C}_{A} - {C}_{B} ) g}{1 + \frac{ψ}{2} g}$$

The difference between the major allele coverage ratio and the minor allele coverage ratio should be zero when the number of copies of the major allele and the minor allele are even (excluding any noise variance), and changes to the copy number should increase at even steps of

$$\frac{g}{1 + \frac{ψ}{2} g} .$$

The sum of the major allele coverage ratio and the minor allele coverage ratio is related to copy number, tumor purity, and ploidy as follows:

$${R}_{A} + {R}_{B} = \frac{2 + ( {C}_{A} + {C}_{B} ) g}{1 + \frac{ψ}{2} g}$$

The minimum value of the sum of the major allele coverage ratio and the minor allele coverage ratio is

$$\frac{2}{1 + \frac{ψ}{2} g} ,$$

and changes to the copy number should increase at even steps of

$$\frac{g}{1 + \frac{ψ}{2} g} .$$

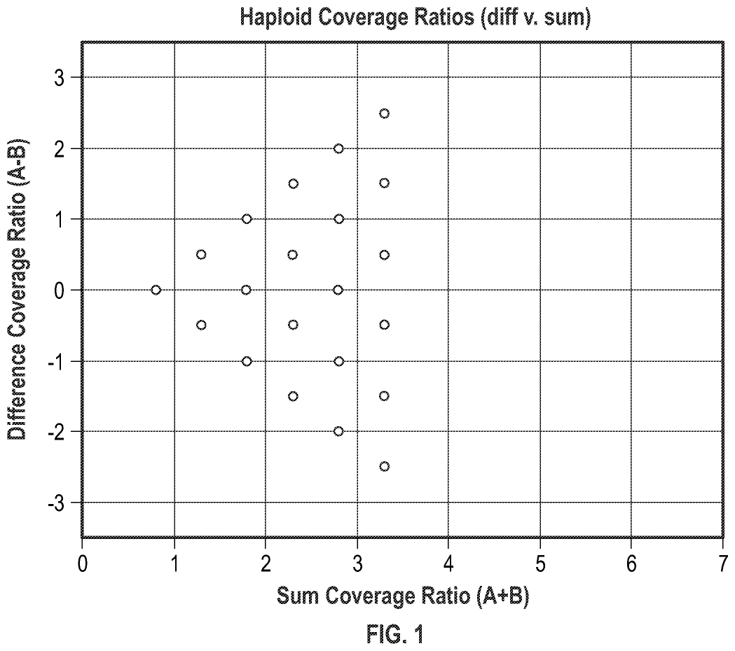

Thus, assuming no noise in the system, each genetic locus should have a transformed data point that lies on one of a set of evenly spaced grid points when the difference between the major allele coverage ratio and the minor allele coverage ratio is plotted against the sum of the major allele coverage ratio and the minor allele coverage ratio. An exemplary set of copy number grid points for a plot of the difference between the major allele coverage ratio and the minor allele coverage ratio against the sum of the major allele coverage ratio and the minor allele coverage ratio is shown in FIG. 1.

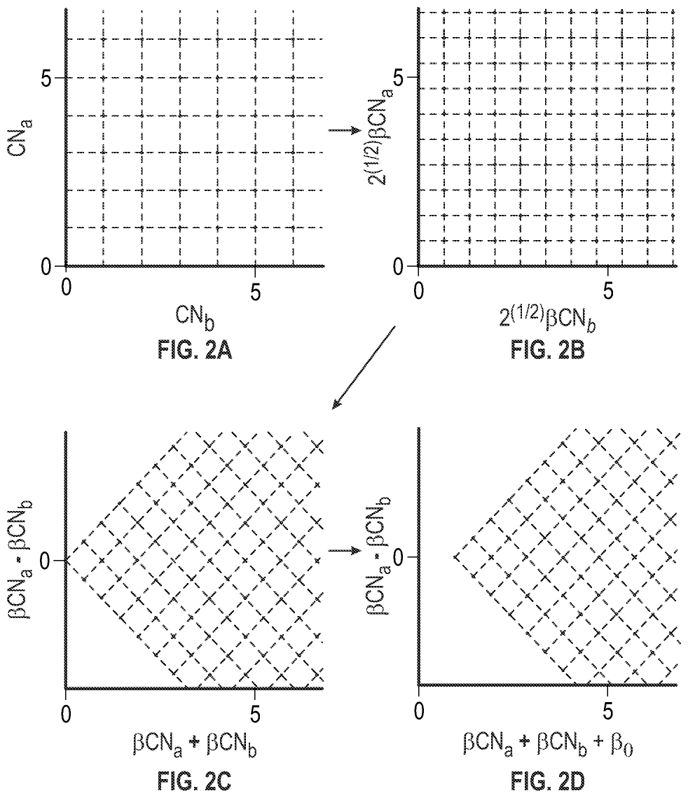

A copy number grid model is representative of copy number space scaled and translated as a function of ploidy and tumor purity values. FIG. 2A shows an exemplary copy number grid in copy number space, wherein the minor allele copy number and the major allele copy number are plotted against each other. Copy numbers are necessarily integer values, so the plot provides an evenly spaced grid. That is, the copy number grid model can comprise allowed copy number states (i.e., integer values for each of the major allele copy number and the minor allele copy number), as represented by the grid points. Copy number grid model parameters β₀ (a translation parameter) and β₁ (a scaling parameter) can be defined as follows:

$$\begin{matrix}{β}_{0} = \frac{2 ( 1 - ρ )}{ψ ρ + 2 ( 1 - ρ )} \\ {β}_{1} = \frac{ρ}{ψ ρ + 2 ( 1 - ρ )}\end{matrix}$$

The scaling copy number grid model parameter can be used to scale the minor allele copy number and the major allele copy number axes. The copy number axes may be scaled by an additional factor, for example \sqrt{2} or 2*\sqrt{2}. Other scaling factors for the copy number axes may be used. FIG. 2B shows an exemplary copy number grid scaled by \sqrt{2}β₁ (assuming a purity of 0.95 and a ploidy of 2 in the illustrated example). The scaled copy number grid can then rotated by

$$θ = - \frac{π}{4} ,$$

as shown in FIG. 2C. Further, the copy number grid may be translated by the translation parameter β₀ as shown in FIG. 2D (in the illustrated example, β₀ was scaled by a factor of 20 to extenuate the separation from the y-axis).

By generating the copy number grid, non-linear parameter space (ρ, ψ) has been transformed to a linear parameter space (β₀, β₁). Further, and likely the more impactful aspect of this representation, is the stability in calling significant copy number events, with respect to degenerate solutions. The copy number grid coordinate system allows β₁ to represent the distance between a neighboring state or a density of states. Further, the copy number grid can establish a “zero level” such that no matter which copy number grid model is fit to the transformed data, β₀ must remain the same for a copy number state having zero copies. The benefit of this transformation is two-fold. The first is that, while there are still two parameters to search, all solutions will contain the same zero-level solution. There is still a degeneracy among the separation of the copy-number events, but the starting point of the grid does not change, only the separation between grid-points. By locking one of the inference parameters into at fixed minimum ratio, the search has been simplified to the separation between states. Further, this system provides a calling method for significant copy number events. Significance can now be defined by the zero level (a fixed parameter), regardless of the separation. Under this methodology, it is no longer necessary to know the copy-number precisely to make a copy number alteration call.

The ploidy and tumor purity values for the sample are unknown apriori. Accordingly, a plurality of copy number grid models can be initialized using different initial tumor purity estimates and tumor ploidy estimates.

In some implementations, initial tumor purity estimates are bound by a preselected initial tumor purity estimate lower limit and/or above a preselected initial tumor purity estimate upper limit. In some implementations, the preselected initial tumor purity estimate lower limit is 0, 0.001, 0.005, 0.01, or any value between these ranges. In some implementations, the preselected initial tumor purity estimate lower limit is 0. In some implementations, the preselected initial tumor purity estimate upper limit is 1, 0.999, 0.995, 0.99, or any value between these ranges. In some implementations, the preselected initial tumor purity estimate upper limit is 1.

In some implementations, initial tumor ploidy estimates are bound by a preselected initial tumor ploidy estimate lower limit and/or above a preselected initial tumor ploidy estimate upper limit. In some implementations, the preselected initial tumor ploidy estimate lower limit is set at about 1.1 to about 1.5, for example about 1.2. In some implementations, the initial tumor ploidy estimate lower limit is 1.2. In some implementations, the preselected initial tumor ploidy estimate upper limit is about 6 to about 10. In some implementations, the preselected initial tumor ploidy estimate upper limit is 8.

Although in some implementations, the initial tumor purity estimates and/or tumor ploidy are bound by physical space (for example, physical tumor purity can never be below 0 or above 1, and the preselected tumor purity estimate lower limit and tumor purity estimate upper limit may be set at or between 0 and 1), the initial or modeled tumor purity estimates and/or tumor ploidy estimates need not be limited to physical space. Due to the model degeneracies, there may exist copy number models that are solvable in in unphysical space (e.g., in which purity>1.0). Although these solutions are unphysical, one could fit models in the unphysical space and use the relationships between degenerate solutions to find the physical values. That is, an unphysical purity can be transformed back to physical purity or physical ploidy. In some embodiments, initial tumor purities and/or initial tumor ploidies for a plurality of copy number grid models may comprise unphysical tumor purity estimates and/or unphysical tumor ploidies. In some embodiments, initial tumor purities and/or initial tumor ploidies for a plurality of copy number grid models may comprise imaginary number tumor purity estimates and/or imaginary number tumor ploidy estimates.

The different copy number grid models can then be fit to the transformed coverage ratio data. By way of example, for any given copy number grid model, the allowed copy number states of the copy number grid model can be fit to the transformed coverage ratio data based on the initial tumor purity estimate and the initial tumor ploidy estimate. A preliminary copy number can be assigned to each genomic segment in the plurality of genomic segment. Genetic loci within a genomic segment are assumed to have the same copy number, although the transformed coverage ratio data of genetic loci within the genomic segment form a 2D Gaussian distribution. The assignment can be based on, for example, the nearest allowed copy number state for the genomic segment. For example, a distance, such as a Mahalanobis distance, from an allowed copy number state may be determined for a genetic segment based on the locations of the transformed coverage ratio data of genetic loci within the genomic segment. Once a preliminary copy number assignment has been made for the genomic segment, there accordingly the genetic loci within the genomic segment, an updated tumor ploidy estimate and updated tumor purity estimate can be determined. The allowed copy number states can then be re-fit to the transformed coverage ratio data based on the updated tumor ploidy estimate and updated tumor purity estimate. Once an updated tumor purity estimate and tumor ploidy estimate is determined, however, the optimal copy number state assignments may no longer be optimal. Thus, the process of assigning a preliminary copy number to each genomic segment in the plurality of genomic segments; determining an updated tumor ploidy estimate and updated tumor purity estimate based on the preliminary copy number assignments; and re-fitting the allowed copy numbers states to the transformed coverage ratio data based on the updated tumor ploidy estimate and updated tumor purity estimate; may be performed iteratively until convergence (i.e., a local fitness maximum).

After fitting a plurality of copy number grid models, a copy number grid model can be selected from the plurality. As discussed above, the transformed coverage ratio data of genetic loci within the genomic segment form a 2D Gaussian distribution, which can be determined. In some implementations of the method, the distance, such as a Mahalanobis distance, for each genomic segment from an assigned copy number state may be assigned. An overall model fit score may be determined based on an average distance between the plurality of genomic segments and the corresponding assigned copy number state. The models may be ranked based on the overall model fit score, although the selected model is not necessarily the model with the best overall model fit score. For example, one or more filters may be applied to the models to exclude certain models. That is, optimal fit alone may not indicate the best model. For example, the models may be filtered by one or more of (i) a preselected ploidy range, (ii) a preselected purity range, (iii) a difference between a noise parameter and copy number state spacing in the model, (iv) models with a ploidy above a preselected value when a corresponding lower-ploidy model is viable, or other desired filtering parameters.

In some implementations, a model may be excluded if the model has a tumor purity below preselected tumor purity lower limit and/or above a preselected tumor purity upper limit. In some implementations, the preselected tumor purity lower limit is 0, 0.001, 0.005, 0.01, or any value between these ranges. In some implementations, the preselected tumor purity lower limit is 0. In some implementations, the preselected tumor purity upper limit is 1, 0.999, 0.995, 0.99, or any value between these ranges. In some implementations, the preselected tumor purity upper limit is 1.

In some implementations, a model may be excluded if the model has a tumor ploidy below preselected tumor ploidy lower limit and/or above a preselected tumor ploidy upper limit. In some implementations, the preselected tumor ploidy lower limit is set at about 1.1 to about 1.5, for example about 1.2. In some implementations, the preselected tumor ploidy lower limit is 1.2. In some implementations, the preselected tumor ploidy upper limit is about 6 to about 10. In some implementations, the preselected tumor ploidy upper limit is 8.

The selected copy number grid model can then be used to assign a copy number state for at least a portion, or all, of the genomic segments. The assigned copy number state may be a total copy number count (i.e., the sum of the major allele copy number and the minor allele copy number), a minor allele copy number count, or a major allele copy number count, for example for a tumor or cancer in the subject. The assignment can be based on, for example, the nearest allowed copy number state for the genomic segment, given a distribution of the transformed coverage ratio data for genetic loci corresponding to the genomic segment. For example, a distance, such as a Mahalanobis distance, from an allowed copy number state may be determined for a genetic segment based on the locations of the transformed coverage ratio data of genetic loci within the genomic segment.

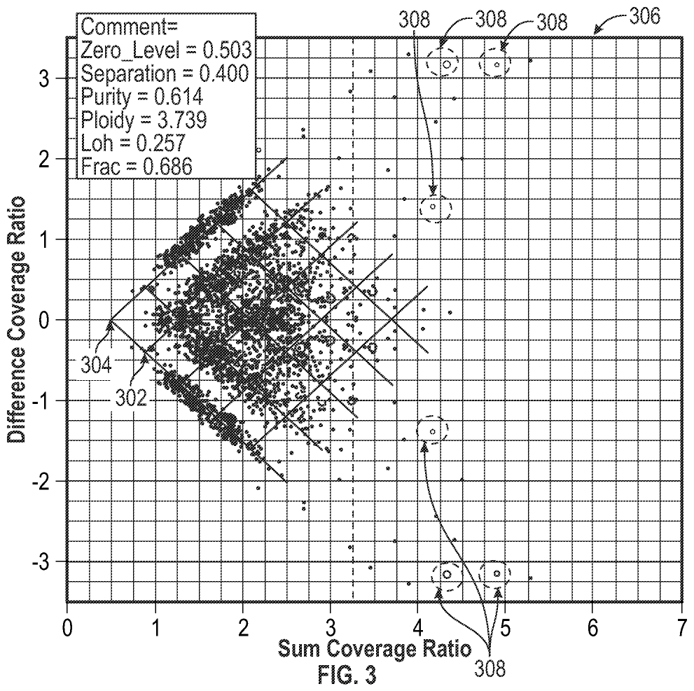

A particular advantage of the method descried herein is that the copy number grid model can be generated (for example, using an interface output on an electronic display) with allele fraction and allele coverage data. Thus, a particular genomic segment or a particular genomic locus can be selected for easy visualization of the copy number state. An exemplary interface that includes a selected copy number grid model with the transformed coverage ratio data is shown in FIG. 3.

In the interface shown in FIG. 3, the transformed coverage ratio data for the plurality of genetic loci is presented as black dots in the plot. The copy number grid model 302 is presented with the transformed coverage ratio data. In a noiseless system, the transformed coverage ratio data would perfectly converge with the grid points (i.e., the intersection of the grid lines), which represent integer copy number states. The spacing of the gridlines and the translocation of the grid depends of the determined purity and ploidy values of 0.614 and 3.739, respectively, in the presented example. Points representing the genomic segments may also be included in the interface, which are optionally proportional to the distribution of transformed coverage ratio data for genomic loci within the genomic segments. This allows for easy visualization of how close the genomic segment copy number state is from an allowed copy number state.

The copy number grid model also facilitates and improves the quality of calling a copy number alteration for one or more genomic loci or one or more genomic segments. In one implementation, the copy number alteration may be called based on the assigned copy number state for the one or more genomic loci or the one or more genomic segments being non-diploid. In one implementation, the copy number alteration may be called based on the assigned copy number state for the one or more genomic loci or the one or more genomic segments being above a predetermined copy number threshold. The predetermined copy number threshold may be selected based on a desired risk tolerance or for a specific gene. For example, certain genes may have a higher predetermined copy number threshold than other genes. In some implementations, a sum coverage ratio (i.e., the sum of the major allele coverage ratio and the minor allele coverage ratio) threshold may be used to call a copy number alteration. An exemplary sum coverage ratio 306 is shown in FIG. 3, set at 3.5. In FIG. 3, genomic segments 308 that exceed the threshold 306 are circled in a dashed line, and a copy number alternation may be called for these genomic segments.

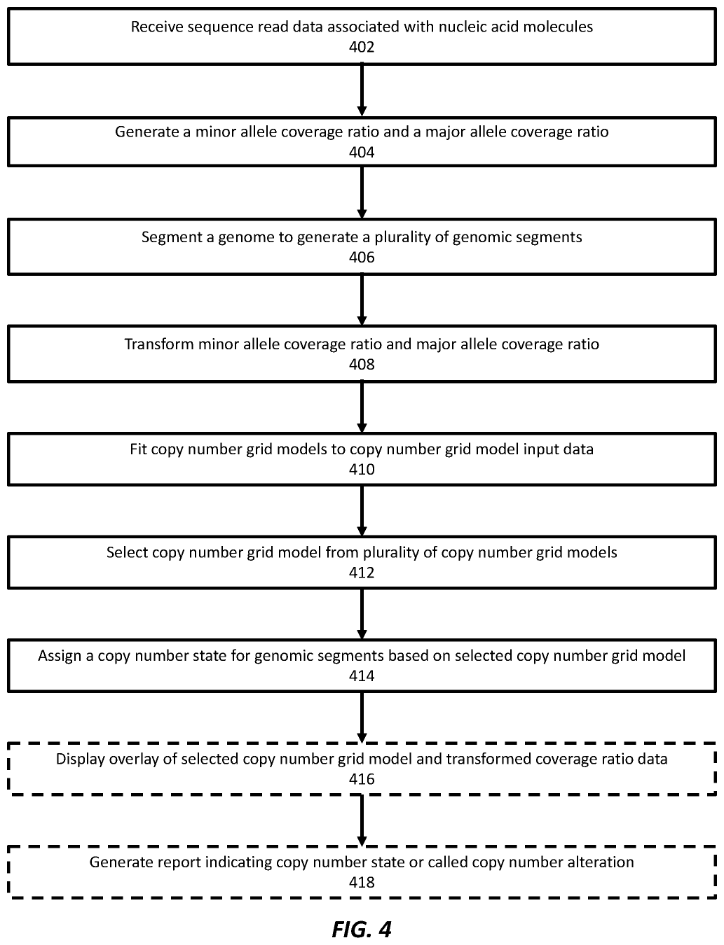

FIG. 4 shows an exemplary method for determining a copy number state for one or more genetic loci or one or more genomic segments. At 402, sequence read data for a plurality of sequence reads are received, for example at one or more processors of an electronic (e.g., computer) system. The sequence read data is associated with a plurality of nucleic acid molecules obtained from a sample from a subject. The sample may include, for example, a mixture of nucleic acid molecules from a tumor tissue and nucleic acid molecules from a non-tumor tissue, or may include cell-free DNA that includes cell-free tumor DNA and cell-free non-tumor DNA. At 404, a minor allele coverage ratio and a major allele coverage ratio are generated for a plurality of genetic loci, for example using the one or more processors. At 406, a genome (e.g., a reference genome appropriate for the subject) is segmented to generate a plurality of genomic segments, for example using the one or more processors. The segmentation may be based on, for example, the minor allele coverage ratio, the major allele coverage ratio, or a total coverage ratio. Segmentation of the genome can identify contiguous portions of the genome that are grouped together based on having similar coverage ratios, and are therefore presumed to have a similar copy number state. Thus, genetic loci within a genome segment can be assumed to have the same copy number state. At 408, the minor allele coverage ratio and the major allele coverage ratio for the genetic loci can be transformed, for example using the one or more processors to generate copy number grid model input data. The copy number grid model input data can include (i) a difference between the major allele coverage ratio and the minor allele coverage ratio, and (i) a sum of the major allele coverage ratio and the minor allele coverage ratio. At 410, a plurality of copy number grid models are fit to the copy number grid model input data. The copy number grid models include allowed copy number states (e.g., integer states), and can be parameterized using tumor purity estimates and tumor ploidy estimates. The different copy number grid models in the plurality of copy number grid models can be initialized using different initial tumor purity estimates and tumor ploidy estimates. At 412, a copy number grid model can be selected from the plurality of copy number grid models. As discussed above, the selected copy number grid model is not necessarily the copy number grid model with the most optimal fit, as one or more copy number grid model filters may be applied to remove unlikely or impossible models. Nevertheless, the copy number grid model selection may be based, at least in part, on an overall model fit score. At 414, a copy number state can be assigned for at least a portion of the genomic segments based on the selected copy number grid model. Optionally, at 416, the selected copy number grid model may be overlaid with the copy number grid model input data, for example via an interface of an electronic display. Also optionally, at 418, a report that indicates the copy number state or a called coy number alteration for one or more genomic loci or one or more genomic segments may be generated. The report may be transmitted to the subject, a healthcare provider, or some other third party, for example via a computer network or a peer-to-peer connection.

In some instances, the disclosed methods may further comprise one or more of the steps of: (i) obtaining the sample from the subject (e.g., a subject suspected of having or determined to have cancer), (ii) extracting nucleic acid molecules (e.g., a mixture of tumor nucleic acid molecules and non-tumor nucleic acid molecules) from the sample, (iii) ligating one or more adapters to the nucleic acid molecules extracted from the sample (e.g., one or more amplification primers, flow cell adaptor sequences, substrate adapter sequences, or sample index sequences), (iv) amplifying the nucleic acid molecules (e.g., using a polymerase chain reaction (PCR) amplification technique, a non-PCR amplification technique, or an isothermal amplification technique), (v) capturing nucleic acid molecules from the amplified nucleic acid molecules (e.g., by hybridization to one or more bait molecules, where the bait molecules each comprise one or more nucleic acid molecules that each comprising a region that is complementary to a region of a captured nucleic acid molecule), (vi) sequencing the nucleic acid molecules extracted from the sample (or library proxies derived therefrom) using, e.g., a next-generation (massively parallel) sequencing technique, a whole genome sequencing (WGS) technique, a whole exome sequencing technique, a targeted sequencing technique, a direct sequencing technique, or a Sanger sequencing technique) using, e.g., a next-generation (massively parallel) sequencer, and (vii) generating, displaying, transmitting, and/or delivering a report (e.g., an electronic, web-based, or paper report) to the subject (or patient), a caregiver, a healthcare provider, a physician, an oncologist, an electronic medical record system, a hospital, a clinic, a third-party payer, an insurance company, or a government office. In some instances, the report comprises output from the methods described herein. In some instances, all or a portion of the report may be displayed in the graphical user interface of an online or web-based healthcare portal. In some instances, the report is transmitted via a computer network or peer-to-peer connection.

The disclosed methods may be used with any of a variety of samples. For example, in some instances, the sample may comprise a tissue biopsy sample, a liquid biopsy sample, or a normal control. In some instances, the sample may be a liquid biopsy sample and may comprise blood, plasma, cerebrospinal fluid, sputum, stool, urine, or saliva. In some instances, the sample may be a liquid biopsy sample and may comprise circulating tumor cells (CTCs). In some instances, the sample may be a liquid biopsy sample and may comprise cell-free DNA (cfDNA), circulating tumor DNA (ctDNA), or any combination thereof.

In some instances, the nucleic acid molecules extracted from a sample may comprise a mixture of tumor nucleic acid molecules and non-tumor nucleic acid molecules. In some instances, the tumor nucleic acid molecules may be derived from a tumor portion of a heterogeneous tissue biopsy sample, and the non-tumor nucleic acid molecules may be derived from a normal portion of the heterogeneous tissue biopsy sample. In some instances, the sample may comprise a liquid biopsy sample, and the tumor nucleic acid molecules may be derived from a circulating tumor DNA (ctDNA) fraction of the liquid biopsy sample while the non-tumor nucleic acid molecules may be derived from a non-tumor, cell-free DNA (cfDNA) fraction of the liquid biopsy sample.

In some instances, the disclosed methods may be used to diagnose the presence of disease (e.g., cancer) in a subject (e.g., a patient). In some instances, the disclosed methods may be applicable to diagnosis of any of a variety of cancers as described elsewhere herein. In some implementations, the disease is a genetic disorder, for example a disease associated with a chromosomal aneuploidy (e.g., Down syndrome, Edwards syndrome, or Patau syndrome) or Fragile X.

In some instances, the disclosed methods may be used to identify a subject as being eligible for a clinical trial for a treatment of a disease. The method may further include enrolling the subject in the clinical trial and/or administering the treatment to the subject. In some instances, the disease may be cancer. In some instances, the disease is a disease associated with a chromosomal aneuploidy (e.g., Down syndrome, Edwards syndrome, or Patau syndrome), or Fragile X.

In some instances, the disclosed methods may be used to select an appropriate therapy or treatment (e.g., a cancer therapy or cancer treatment) for a subject. In some instances, for example, the cancer therapy or treatment may comprise use of a poly (ADP-ribose) polymerase inhibitor (PARPi), a platinum compound, chemotherapy, radiation therapy, a targeted therapy (e.g., immunotherapy), surgery, or any combination thereof.

In some instances, the disclosed methods may be used in treating a disease (e.g., a cancer) in a subject. For example, in response to determining that CNAs occur in one or more gene loci in a patient sample using any of the methods disclosed herein, an effective amount of a cancer therapy or cancer treatment may be administered to the subject.

In some instances, the disclosed methods may be used for monitoring disease progression or recurrence (e.g., cancer or tumor progression or recurrence) in a subject. For example, in some instances, the methods may be used to detect CNAs in a first sample obtained from the subject at a first time point, and used to detect CNAs in a second sample obtained from the subject at a second time point, where comparison of the first determination of CNAs and the second determination of CNAs allows one to monitor disease progression or recurrence. In some instances, the first time point is chosen before the subject has been administered a therapy or treatment, and the second time point is chosen after the subject has been administered the therapy or treatment.

In some instances, the disclosed methods may be used to select a patient for a clinical trial. For example, the patient may be selected based on having a copy number alteration in one or more genes called using the methods described herein.

In some instances, the disclosed methods may be used for adjusting a therapy or treatment (e.g., a cancer treatment or cancer therapy) for a subject, e.g., by adjusting a treatment dose and/or selecting a different treatment in response to a change in called copy number alterations (CNAs).

In some instances, called CNAs determined using the disclosed methods may be used as a prognostic or diagnostic indicator associated with the sample. For example, in some instances, the prognostic or diagnostic indicator may comprise an indicator of the presence of a disease (e.g., cancer) in the sample, an indicator of the probability that a disease (e.g., cancer) is present in the sample, an indicator of the probability that the subject from which the sample was derived will develop a disease (e.g., cancer) (i.e., a risk factor), or an indicator of the likelihood that the subject from which the sample was derived will respond to a particular therapy or treatment.

In some instances, the disclosed methods may be implemented as part of a genomic profiling process that comprises identification of the presence of variant sequences at one or more gene loci in a sample derived from a subject as part of detecting, monitoring, predicting a risk factor, or selecting a treatment for a particular disease, e.g., cancer. In some instances, the variant panel selected for genomic profiling may comprise the detection of variant sequences at a selected set of gene loci. In some instances, the variant panel selected for genomic profiling may comprise detection of variant sequences at a number of gene loci through comprehensive genomic profiling (CGP), a next-generation sequencing (NGS) approach used to assess hundreds of genes (including relevant cancer biomarkers) in a single assay. Inclusion of the disclosed methods as part of a genomic profiling process (or inclusion of the output from the disclosed methods for called CNAs as part of the genomic profile of the subject) can improve the validity of, e.g., disease detection calls and treatment decisions, made on the basis of the genomic profile by, for example, independently confirming the presence of CNAs in one or more gene loci in a given patient sample.

In some instances, a genomic profile may comprise information on the presence of genes (or variant sequences thereof), copy number variations, epigenetic traits, proteins (or modifications thereof), and/or other biomarkers in an individual's genome and/or proteome, as well as information on the individual's corresponding phenotypic traits and the interaction between genetic or genomic traits, phenotypic traits, and environmental factors.

In some instances, a genomic profile for the subject may comprise results from a comprehensive genomic profiling (CGP) test, a nucleic acid sequencing-based test, a gene expression profiling test, a cancer hotspot panel test, a DNA methylation test, a DNA fragmentation test, an RNA fragmentation test, or any combination thereof.

In some instances, the method can further include administering or applying a treatment or therapy (e.g., an anti-cancer agent, anti-cancer treatment, or anti-cancer therapy) to the subject based on the generated genomic profile. An anti-cancer agent or anti-cancer treatment may refer to a compound that is effective in the treatment of cancer cells. Examples of anti-cancer agents or anti-cancer therapies include, but not limited to, alkylating agents, antimetabolites, natural products, hormones, chemotherapy, radiation therapy, immunotherapy, surgery, or a therapy configured to target a defect in a specific cell-signaling pathway, e.g., a defect in a DNA mismatch repair (MMR) pathway.

### Samples

The disclosed methods and systems may be used with any of a variety of samples (also referred to herein as specimens) comprising nucleic acids (e.g., DNA or RNA) that are collected from a subject (e.g., a patient). Examples include, but are not limited to, a tumor sample, a tissue sample, a biopsy sample, a blood sample (e.g., a peripheral whole blood sample), a blood plasma sample, a blood serum sample, a lymph sample, a saliva sample, a sputum sample, a urine sample, a gynecological fluid sample, a circulating tumor cell (CTC) sample, a cerebral spinal fluid (CSF) sample, a pericardial fluid sample, a pleural fluid sample, an ascites (peritoneal fluid) sample, a feces (or stool) sample, or other body fluid, secretion, and/or excretion sample (or cell sample derived therefrom). In certain instances, the sample may be frozen sample or a formalin-fixed paraffin-embedded (FFPE) sample.

In some instances, the sample may be collected by tissue resection (e.g., surgical resection), needle biopsy, bone marrow biopsy, bone marrow aspiration, skin biopsy, endoscopic biopsy, fine needle aspiration, oral swab, nasal swab, vaginal swab or a cytology smear, scrapings, washings or lavages (such as a ductal lavages or bronchoalveolar lavages), etc.

In some instances, the sample is a liquid biopsy sample, and may comprise, e.g., whole blood, blood plasma, blood serum, urine, stool, sputum, saliva, or cerebrospinal fluid. In some instances, the sample may be a liquid biopsy sample and may comprise circulating tumor cells (CTCs). In some instances, the sample may be a liquid biopsy sample and may comprise cell-free DNA (cfDNA), circulating tumor DNA (ctDNA), or any combination thereof.

In some instances, the sample may comprise one or more premalignant or malignant cells. Premalignant, as used herein, refers to a cell or tissue that is not yet malignant but is poised to become malignant. In certain instances, the sample may be acquired from a solid tumor, a soft tissue tumor, or a metastatic lesion. In certain instances, the sample may be acquired from a hematologic malignancy or pre-malignancy. In other instances, the sample may comprise a tissue or cells from a surgical margin. In certain instances, the sample may comprise tumor-infiltrating lymphocytes. In some instances, the sample may comprise one or more non-malignant cells. In some instances, the sample may be, or is part of, a primary tumor or a metastasis (e.g., a metastasis biopsy sample). In some instances, the sample may be obtained from a site (e.g., a tumor site) with the highest percentage of tumor (e.g., tumor cells) as compared to adjacent sites (e.g., sites adjacent to the tumor). In some instances, the sample may be obtained from a site (e.g., a tumor site) with the largest tumor focus (e.g., the largest number of tumor cells as visualized under a microscope) as compared to adjacent sites (e.g., sites adjacent to the tumor).

In some instances, the disclosed methods may further comprise analyzing a primary control (e.g., a normal tissue sample). In some instances, the disclosed methods may further comprise determining if a primary control is available and, if so, isolating a control nucleic acid (e.g., DNA) from said primary control. In some instances, the sample may comprise any normal control (e.g., a normal adjacent tissue (NAT)) if no primary control is available. In some instances, the sample may be or may comprise histologically normal tissue. In some instances, the method includes evaluating a sample, e.g., a histologically normal sample (e.g., from a surgical tissue margin) using the methods described herein. In some instances, the disclosed methods may further comprise acquiring a sub-sample enriched for non-tumor cells, e.g., by macro-dissecting non-tumor tissue from said NAT in a sample not accompanied by a primary control. In some instances, the disclosed methods may further comprise determining that no primary control and no NAT is available, and marking said sample for analysis without a matched control.

In some instances, samples obtained from histologically normal tissues (e.g., otherwise histologically normal surgical tissue margins) may still comprise a genetic alteration such as a variant sequence as described herein. The methods may thus further comprise re-classifying a sample based on the presence of the detected genetic alteration. In some instances, multiple samples (e.g., from different subjects) are processed simultaneously.

The disclosed methods and systems may be applied to the analysis of nucleic acids extracted from any of variety of tissue samples (or disease states thereof), e.g., solid tissue samples, soft tissue samples, metastatic lesions, or liquid biopsy samples. Examples of tissues include, but are not limited to, connective tissue, muscle tissue, nervous tissue, epithelial tissue, and blood. Tissue samples may be collected from any of the organs within an animal or human body. Examples of human organs include, but are not limited to, the brain, heart, lungs, liver, kidneys, pancreas, spleen, thyroid, mammary glands, uterus, prostate, large intestine, small intestine, bladder, bone, skin, etc.

In some instances, the nucleic acids extracted from the sample may comprise deoxyribonucleic acid (DNA) molecules. Examples of DNA that may be suitable for analysis by the disclosed methods include, but are not limited to, genomic DNA or fragments thereof, mitochondrial DNA or fragments thereof, cell-free DNA (cfDNA), and circulating tumor DNA (ctDNA). Cell-free DNA (cfDNA) is comprised of fragments of DNA that are released from normal and/or cancerous cells during apoptosis and necrosis, and circulate in the blood stream and/or accumulate in other bodily fluids. Circulating tumor DNA (ctDNA) is comprised of fragments of DNA that are released from cancerous cells and tumors that circulate in the blood stream and/or accumulate in other bodily fluids.

In some instances, DNA is extracted from nucleated cells from the sample. In some instances, a sample may have a low nucleated cellularity, e.g., when the sample is comprised mainly of erythrocytes, lesional cells that contain excessive cytoplasm, or tissue with fibrosis. In some instances, a sample with low nucleated cellularity may require more, e.g., greater, tissue volume for DNA extraction.

In some instances, the nucleic acids extracted from the sample may comprise ribonucleic acid (RNA) molecules. Examples of RNA that may be suitable for analysis by the disclosed methods include, but are not limited to, total cellular RNA, total cellular RNA after depletion of certain abundant RNA sequences (e.g., ribosomal RNAs), cell-free RNA (cfRNA), messenger RNA (mRNA) or fragments thereof, the poly(A)-tailed mRNA fraction of the total RNA, ribosomal RNA (rRNA) or fragments thereof, transfer RNA (tRNA) or fragments thereof, and mitochondrial RNA or fragments thereof. In some instances, RNA may be extracted from the sample and converted to complementary DNA (cDNA) using, e.g., a reverse transcription reaction. In some instances, the cDNA is produced by random-primed cDNA synthesis methods. In other instances, the cDNA synthesis is initiated at the poly(A) tail of mature mRNAs by priming with oligo(dT)-containing oligonucleotides. Methods for depletion, poly(A) enrichment, and cDNA synthesis are well known to those of skill in the art.

In some instances, the sample may comprise a tumor content, e.g., comprising tumor cells or tumor cell nuclei. In some instances, the sample may comprise a tumor content of at least 5-50%, 10-40%, 15-25%, or 20-30% tumor cell nuclei. In some instances, the sample may comprise a tumor content of at least 5%, at least 10%, at least 20%, at least 30%, at least 40%, or at least 50% tumor cell nuclei. In some instances, the percent tumor cell nuclei is determined (e.g., calculated) by dividing the number of tumor cells in the sample by the total number of all cells within the sample that have nuclei. In some instances, for example when the sample is a liver sample comprising hepatocytes, a different tumor content calculation may be required due to the presence of hepatocytes having nuclei with twice, or more than twice, the DNA content of other, e.g., non-hepatocyte, somatic cell nuclei. In some instances, the sensitivity of detection of a genetic alteration, e.g., a variant sequence, or a determination of, e.g., microsatellite instability, may depend on the tumor content of the sample. For example, a sample having a lower tumor content can result in lower sensitivity of detection for a given size sample.

In some instances, as noted above, the sample comprises nucleic acid (e.g., DNA, RNA (or a cDNA derived from the RNA), or both), e.g., from a tumor or from normal tissue. In certain instances, the sample may further comprise a non-nucleic acid component, e.g., cells, protein, carbohydrate, or lipid, e.g., from the tumor or normal tissue.

### Subjects

In some instances, the sample is obtained (e.g., collected) from a subject (e.g., patient) with a condition or disease (e.g., a hyperproliferative disease or a non-cancer indication) or suspected of having the condition or disease. In some instances, the hyperproliferative disease is a cancer. In some instances, the cancer is a solid tumor or a metastatic form thereof. In some instances, the cancer is a hematological cancer, e.g. a leukemia or lymphoma.

In some instances, the subject has a cancer or is at risk of having a cancer. For example, in some instances, the subject has a genetic predisposition to a cancer (e.g., having a genetic mutation that increases his or her baseline risk for developing a cancer). In some instances, the subject has been exposed to an environmental perturbation (e.g., radiation or a chemical) that increases his or her risk for developing a cancer. In some instances, the subject is in need of being monitored for development of a cancer. In some instances, the subject is in need of being monitored for cancer progression or regression, e.g., after being treated with a cancer therapy (or cancer treatment). In some instances, the subject is in need of being monitored for relapse of cancer. In some instances, the subject is in need of being monitored for minimum residual disease (MRD). In some instances, the subject has been, or is being treated, for cancer. In some instances, the subject has not been treated with a cancer therapy (or cancer treatment).

In some instances, the subject (e.g., a patient) is being treated, or has been previously treated, with one or more targeted therapies. In some instances, e.g., for a patient who has been previously treated with a targeted therapy, a post-targeted therapy sample (e.g., specimen) is obtained (e.g., collected). In some instances, the post-targeted therapy sample is a sample obtained after the completion of the targeted therapy.

In some instances, the patient has not been previously treated with a targeted therapy. In some instances, e.g., for a patient who has not been previously treated with a targeted therapy, the sample comprises a resection, e.g., an original resection, or a resection following recurrence (e.g., following a disease recurrence post-therapy).

### Cancers

In some instances, the sample is acquired from a subject having a cancer. Exemplary cancers include, but are not limited to, B cell cancer (e.g., multiple myeloma), melanomas, breast cancer, lung cancer (such as non-small cell lung carcinoma or NSCLC), bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, adenocarcinomas, inflammatory myofibroblastic tumors, gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancers, essential thrombocythemia, agnogenic myeloid metaplasia, hypereosinophilic syndrome, systemic mastocytosis, familiar hypereosinophilia, chronic eosinophilic leukemia, neuroendocrine cancers, carcinoid tumors, and the like.

In some instances, the cancer is a hematologic malignancy (or premaligancy). As used herein, a hematologic malignancy refers to a tumor of the hematopoietic or lymphoid tissues, e.g., a tumor that affects blood, bone marrow, or lymph nodes. Exemplary hematologic malignancies include, but are not limited to, leukemia (e.g., acute lymphoblastic leukemia (ALL), acute myeloid leukemia (AML), chronic lymphocytic leukemia (CLL), chronic myelogenous leukemia (CML), hairy cell leukemia, acute monocytic leukemia (AMoL), chronic myelomonocytic leukemia (CMML), juvenile myelomonocytic leukemia (JMML), or large granular lymphocytic leukemia), lymphoma (e.g., AIDS-related lymphoma, cutaneous T-cell lymphoma, Hodgkin lymphoma (e.g., classical Hodgkin lymphoma or nodular lymphocyte-predominant Hodgkin lymphoma), mycosis fungoides, non-Hodgkin lymphoma (e.g., B-cell non-Hodgkin lymphoma (e.g., Burkitt lymphoma, small lymphocytic lymphoma (CLL/SLL), diffuse large B-cell lymphoma, follicular lymphoma, immunoblastic large cell lymphoma, precursor B-lymphoblastic lymphoma, or mantle cell lymphoma) or T-cell non-Hodgkin lymphoma (mycosis fungoides, anaplastic large cell lymphoma, or precursor T-lymphoblastic lymphoma)), primary central nervous system lymphoma, Sezary syndrome, Waldenstrom macroglobulinemia), chronic myeloproliferative neoplasm, Langerhans cell histiocytosis, multiple myeloma/plasma cell neoplasm, myelodysplastic syndrome, or myelodysplastic/myeloproliferative neoplasm.

### Nucleic Acid Extraction and Processing

DNA or RNA may be extracted from tissue samples, biopsy samples, blood samples, or other bodily fluid samples using any of a variety of techniques known to those of skill in the art (see, e.g., Example 1 of International Patent Application Publication No. WO 2012/092426; Tan, et al. (2009), “DNA, RNA, and Protein Extraction: The Past and The Present”, J. Biomed. Biotech. 2009:574398; the technical literature for the Maxwell® 16 LEV Blood DNA Kit (Promega Corporation, Madison, WI); and the Maxwell 16 Buccal Swab LEV DNA Purification Kit Technical Manual (Promega Literature #TM333, Jan. 1, 2011, Promega Corporation, Madison, WI)). Protocols for RNA isolation are disclosed in, e.g., the Maxwell® 16 Total RNA Purification Kit Technical Bulletin (Promega Literature #TB351, August 2009, Promega Corporation, Madison, WI).

A typical DNA extraction procedure, for example, comprises (i) collection of the fluid sample, cell sample, or tissue sample from which DNA is to be extracted, (ii) disruption of cell membranes (i.e., cell lysis), if necessary, to release DNA and other cytoplasmic components, (iii) treatment of the fluid sample or lysed sample with a concentrated salt solution to precipitate proteins, lipids, and RNA, followed by centrifugation to separate out the precipitated proteins, lipids, and RNA, and (iv) purification of DNA from the supernatant to remove detergents, proteins, salts, or other reagents used during the cell membrane lysis step.

Disruption of cell membranes may be performed using a variety of mechanical shear (e.g., by passing through a French press or fine needle) or ultrasonic disruption techniques. The cell lysis step often comprises the use of detergents and surfactants to solubilize lipids the cellular and nuclear membranes. In some instances, the lysis step may further comprise use of proteases to break down protein, and/or the use of an RNase for digestion of RNA in the sample.

Examples of suitable techniques for DNA purification include, but are not limited to, (i) precipitation in ice-cold ethanol or isopropanol, followed by centrifugation (precipitation of DNA may be enhanced by increasing ionic strength, e.g., by addition of sodium acetate), (ii) phenol-chloroform extraction, followed by centrifugation to separate the aqueous phase containing the nucleic acid from the organic phase containing denatured protein, and (iii) solid phase chromatography where the nucleic acids adsorb to the solid phase (e.g., silica or other) depending on the pH and salt concentration of the buffer.

In some instances, cellular and histone proteins bound to the DNA may be removed either by adding a protease or by having precipitated the proteins with sodium or ammonium acetate, or through extraction with a phenol-chloroform mixture prior to a DNA precipitation step.

In some instances, DNA may be extracted using any of a variety of suitable commercial DNA extraction and purification kits. Examples include, but are not limited to, the QIAamp (for isolation of genomic DNA from human samples) and DNAeasy (for isolation of genomic DNA from animal or plant samples) kits from Qiagen (Germantown, MD) or the Maxwell® and ReliaPrep™ series of kits from Promega (Madison, WI).

As noted above, in some instances the sample may comprise a formalin-fixed (also known as formaldehyde-fixed, or paraformaldehyde-fixed), paraffin-embedded (FFPE) tissue preparation. For example, the FFPE sample may be a tissue sample embedded in a matrix, e.g., an FFPE block. Methods to isolate nucleic acids (e.g., DNA) from formaldehyde- or paraformaldehyde-fixed, paraffin-embedded (FFPE) tissues are disclosed in, e.g., Cronin, et al., (2004) Am J Pathol. 164(1):35-42; Masuda, et al., (1999) Nucleic Acids Res. 27(22):4436-4443; Specht, et al., (2001) Am J Pathol. 158(2):419-429; the Ambion RecoverAll™ Total Nucleic Acid Isolation Protocol (Ambion, Cat. No. AM1975, September 2008); the Maxwell® 16 FFPE Plus LEV DNA Purification Kit Technical Manual (Promega Literature #TM349, February 2011); the E.Z.N.A.® FFPE DNA Kit Handbook (OMEGA bio-tek, Norcross, GA, product numbers D3399-00, D3399-01, and D3399-02, June 2009); and the QIAamp® DNA FFPE Tissue Handbook (Qiagen, Cat. No. 37625, October 2007). For example, the RecoverAll™ Total Nucleic Acid Isolation Kit uses xylene at elevated temperatures to solubilize paraffin-embedded samples and a glass-fiber filter to capture nucleic acids. The Maxwell® 16 FFPE Plus LEV DNA Purification Kit is used with the Maxwell® 16 Instrument for purification of genomic DNA from 1 to 10 μm sections of FFPE tissue. DNA is purified using silica-clad paramagnetic particles (PMPs), and eluted in low elution volume. The E.Z.N.A.® FFPE DNA Kit uses a spin column and buffer system for isolation of genomic DNA. QIAamp® DNA FFPE Tissue Kit uses QIAamp® DNA Micro technology for purification of genomic and mitochondrial DNA.

In some instances, the disclosed methods may further comprise determining or acquiring a yield value for the nucleic acid extracted from the sample and comparing the determined value to a reference value. For example, if the determined or acquired value is less than the reference value, the nucleic acids may be amplified prior to proceeding with library construction. In some instances, the disclosed methods may further comprise determining or acquiring a value for the size (or average size) of nucleic acid fragments in the sample, and comparing the determined or acquired value to a reference value, e.g., a size (or average size) of at least 100, 200, 300, 400, 500, 600, 700, 800, 900, or 1000 base pairs (bps). In some instances, one or more parameters described herein may be adjusted or selected in response to this determination.

After isolation, the nucleic acids are typically dissolved in a slightly alkaline buffer, e.g., Tris-EDTA (TE) buffer, or in ultra-pure water. In some instances, the isolated nucleic acids (e.g., genomic DNA) may be fragmented or sheared by using any of a variety of techniques known to those of skill in the art. For example, genomic DNA can be fragmented by physical shearing methods, enzymatic cleavage methods, chemical cleavage methods, and other methods known to those of skill in the art. Methods for DNA shearing are described in Example 4 in International Patent Application Publication No. WO 2012/092426. In some instances, alternatives to DNA shearing methods can be used to avoid a ligation step during library preparation.

### Library Preparation

In some instances, the nucleic acids isolated from the sample may be used to construct a library (e.g., a nucleic acid library as described herein). In some instances, the nucleic acids are fragmented using any of the methods described above, optionally subjected to repair of chain end damage, and optionally ligated to synthetic adapters, primers, and/or barcodes (e.g., amplification primers, sequencing adapters, flow cell adapters, substrate adapters, sample barcodes or indexes, and/or unique molecular identifier sequences), size-selected (e.g., by preparative gel electrophoresis), and/or amplified (e.g., using PCR, a non-PCR amplification technique, or an isothermal amplification technique). In some instances, the fragmented and adapter-ligated group of nucleic acids is used without explicit size selection or amplification prior to hybridization-based selection of target sequences. In some instances, the nucleic acid is amplified by any of a variety of specific or non-specific nucleic acid amplification methods known to those of skill in the art. In some instances, the nucleic acids are amplified, e.g., by a whole-genome amplification method such as random-primed strand-displacement amplification. Examples of nucleic acid library preparation techniques for next-generation sequencing are described in, e.g., van Dijk, et al. (2014), Exp. Cell Research 322:12-20, and Illumina's genomic DNA sample preparation kit.

In some instances, the resulting nucleic acid library may contain all or substantially all of the complexity of the genome. The term “substantially all” in this context refers to the possibility that there can in practice be some unwanted loss of genome complexity during the initial steps of the procedure. The methods described herein also are useful in cases where the nucleic acid library comprises a portion of the genome, e.g., where the complexity of the genome is reduced by design. In some instances, any selected portion of the genome can be used with a method described herein. For example, in certain embodiments, the entire exome or a subset thereof is isolated. In some instances, the library may include at least 95%, 90%, 80%, 70%, 60%, 50%, 40%, 30%, 20%, 10%, or 5% of the genomic DNA. In some instances, the library may consist of cDNA copies of genomic DNA that includes copies of at least 95%, 90%, 80%, 70%, 60%, 50%, 40%, 30%, 20%, 10%, or 5% of the genomic DNA. In certain instances, the amount of nucleic acid used to generate the nucleic acid library may be less than 5 micrograms, less than 1 microgram, less than 500 ng, less than 200 ng, less than 100 ng, less than 50 ng, less than 10 ng, less than 5 ng, or less than 1 ng.

In some instances, a library (e.g., a nucleic acid library) includes a collection of nucleic acid molecules. As described herein, the nucleic acid molecules of the library can include a target nucleic acid molecule (e.g., a tumor nucleic acid molecule, a reference nucleic acid molecule and/or a control nucleic acid molecule; also referred to herein as a first, second and/or third nucleic acid molecule, respectively). The nucleic acid molecules of the library can be from a single subject or individual. In some instances, a library can comprise nucleic acid molecules derived from more than one subject (e.g., 2, 3, 4, 5, 6, 7, 8, 9, 10, 20, 30 or more subjects). For example, two or more libraries from different subjects can be combined to form a library having nucleic acid molecules from more than one subject (where the nucleic acid molecules derived from each subject are optionally ligated to a unique sample barcode corresponding to a specific subject). In some instances, the subject is a human having, or at risk of having, a cancer or tumor.

In some instances, the library (or a portion thereof) may comprise one or more subgenomic intervals. In some instances, a subgenomic interval can be a single nucleotide position, e.g., a nucleotide position for which a variant at the position is associated (positively or negatively) with a tumor phenotype. In some instances, a subgenomic interval comprises more than one nucleotide position. Such instances include sequences of at least 2, 5, 10, 50, 100, 150, 250, or more than 250 nucleotide positions in length. Subgenomic intervals can comprise, e.g., one or more entire genes (or portions thereof), one or more exons or coding sequences (or portions thereof), one or more introns (or portion thereof), one or more microsatellite region (or portions thereof), or any combination thereof. A subgenomic interval can comprise all or a part of a fragment of a naturally occurring nucleic acid molecule, e.g., a genomic DNA molecule. For example, a subgenomic interval can correspond to a fragment of genomic DNA which is subjected to a sequencing reaction. In some instances, a subgenomic interval is a continuous sequence from a genomic source. In some instances, a subgenomic interval includes sequences that are not contiguous in the genome, e.g., subgenomic intervals in cDNA can include exon-exon junctions formed as a result of splicing. In some instances, the subgenomic interval comprises a tumor nucleic acid molecule. In some instances, the subgenomic interval comprises a non-tumor nucleic acid molecule.

### Targeting Gene Loci for Analysis

The methods described herein can be used in combination with, or as part of, a method for evaluating a plurality or set of subject intervals (e.g., target sequences), e.g., from a set of genomic loci (e.g., gene loci or fragments thereof), as described herein.

In some instances, the set of genomic loci evaluated by the disclosed methods comprises a plurality of, e.g., genes, which in mutant form, are associated with an effect on cell division, growth or survival, or are associated with a cancer, e.g., a cancer described herein.

In some instances, the set of gene loci evaluated by the disclosed methods comprises at least 1, at least 2, at least 3, at least 4, at least 5, at least 6, at least 7, at least 8, at least 9, at least 10, at least 20, at least 30, at least 40, at least 50, at least 60, at least 70, at least 80, at least 90, at least 100, or more than 100 gene loci.

In some instances, the selected gene loci (also referred to herein as target gene loci or target sequences), or fragments thereof, may include subject intervals comprising non-coding sequences, coding sequences, intragenic regions, or intergenic regions of the subject genome. For example, the subject intervals can include a non-coding sequence or fragment thereof (e.g., a promoter sequence, enhancer sequence, 5′ untranslated region (5′ UTR), 3′ untranslated region (3′ UTR), or a fragment thereof), a coding sequence of fragment thereof, an exon sequence or fragment thereof, an intron sequence or a fragment thereof.

### Target Capture Reagents

The methods described herein may comprise contacting a nucleic acid library with a plurality of target capture reagents in order to select and capture a plurality of specific target sequences (e.g., gene sequences or fragments thereof) for analysis. In some instances, a target capture reagent (i.e., a molecule which can bind to and thereby allow capture of a target molecule) is used to select the subject intervals to be analyzed. For example, a target capture reagent can be a bait molecule, e.g., a nucleic acid molecule (e.g., a DNA molecule or RNA molecule) which can hybridize to (i.e., is complementary to) a target molecule, and thereby allows capture of the target nucleic acid. In some instances, the target capture reagent, e.g., a bait molecule (or bait sequence), is a capture oligonucleotide (or capture probe). In some instances, the target nucleic acid is a genomic DNA molecule, an RNA molecule, a cDNA molecule derived from an RNA molecule, a microsatellite DNA sequence, and the like. In some instances, the target capture reagent is suitable for solution-phase hybridization to the target. In some instances, the target capture reagent is suitable for solid-phase hybridization to the target. In some instances, the target capture reagent is suitable for both solution-phase and solid-phase hybridization to the target. The design and construction of target capture reagents is described in more detail in, e.g., International Patent Application Publication No. WO 2020/236941, the entire content of which is incorporated herein by reference.

The methods described herein provide for optimized sequencing of a large number of genomic loci (e.g., genes or gene products (e.g., mRNA), microsatellite loci, etc.) from samples (e.g., cancerous tissue specimens, liquid biopsy samples, and the like) from one or more subjects by the appropriate selection of target capture reagents to select the target nucleic acid molecules to be sequenced. In some instances, a target capture reagent may hybridize to a specific target locus, e.g., a specific target gene locus or fragment thereof. In some instances, a target capture reagent may hybridize to a specific group of target loci, e.g., a specific group of gene loci or fragments thereof. In some instances, a plurality of target capture reagents comprising a mix of target-specific and/or group-specific target capture reagents may be used.

In some instances, the number of target capture reagents (e.g., bait molecules) in the plurality of target capture reagents (e.g., a bait set) contacted with a nucleic acid library to capture a plurality of target sequences for nucleic acid sequencing is greater than 10, greater than 50, greater than 100, greater than 200, greater than 300, greater than 400, greater than 500, greater than 600, greater than 700, greater than 800, greater than 900, greater than 1,000, greater than 1,250, greater than 1,500, greater than 1,750, greater than 2,000, greater than 3,000, greater than 4,000, greater than 5,000, greater than 10,000, greater than 25,000, or greater than 50,000.

In some instances, the overall length of the target capture reagent sequence can be between about 70 nucleotides and 1000 nucleotides. In one instance, the target capture reagent length is between about 100 and 300 nucleotides, 110 and 200 nucleotides, or 120 and 170 nucleotides, in length. In addition to those mentioned above, intermediate oligonucleotide lengths of about 70, 80, 90, 100, 110, 120, 130, 140, 150, 160, 170, 180, 190, 200, 210, 220, 230, 240, 250, 300, 400, 500, 600, 700, 800, and 900 nucleotides in length can be used in the methods described herein. In some embodiments, oligonucleotides of about 70, 80, 90, 100, 110, 120, 130, 140, 150, 160, 170, 180, 190, 200, 210, 220, or 230 bases can be used.

In some instances, each target capture reagent sequence can include: (i) a target-specific capture sequence (e.g., a gene locus or microsatellite locus-specific complementary sequence), (ii) an adapter, primer, barcode, and/or unique molecular identifier sequence, and (iii) universal tails on one or both ends. As used herein, the term “target capture reagent” can refer to the target-specific target capture sequence or to the entire target capture reagent oligonucleotide including the target-specific target capture sequence.

In some instances, the target-specific capture sequences in the target capture reagents are between about 40 nucleotides and 1000 nucleotides in length. In some instances, the target-specific capture sequence is between about 70 nucleotides and 300 nucleotides in length. In some instances, the target-specific sequence is between about 100 nucleotides and 200 nucleotides in length. In yet other instances, the target-specific sequence is between about 120 nucleotides and 170 nucleotides in length, typically 120 nucleotides in length. Intermediate lengths in addition to those mentioned above also can be used in the methods described herein, such as target-specific sequences of about 40, 50, 60, 70, 80, 90, 100, 110, 120, 130, 140, 150, 160, 170, 180, 190, 200, 210, 220, 230, 240, 250, 300, 400, 500, 600, 700, 800, and 900 nucleotides in length, as well as target-specific sequences of lengths between the above-mentioned lengths.

In some instances, the target capture reagent may be designed to select a subject interval containing one or more rearrangements, e.g., an intron containing a genomic rearrangement. In such instances, the target capture reagent is designed such that repetitive sequences are masked to increase the selection efficiency. In those instances where the rearrangement has a known juncture sequence, complementary target capture reagents can be designed to recognize the juncture sequence to increase the selection efficiency.

In some instances, the disclosed methods may comprise the use of target capture reagents designed to capture two or more different target categories, each category having a different target capture reagent design strategy. In some instances, the hybridization-based capture methods and target capture reagent compositions disclosed herein may provide for the capture and homogeneous coverage of a set of target sequences, while minimizing coverage of genomic sequences outside of the targeted set of sequences. In some instances, the target sequences may include the entire exome of genomic DNA or a selected subset thereof. In some instances, the target sequences may include, e.g., a large chromosomal region (e.g., a whole chromosome arm). The methods and compositions disclosed herein provide different target capture reagents for achieving different sequencing depths and patterns of coverage for complex sets of target nucleic acid sequences.

Typically, DNA molecules are used as target capture reagent sequences, although RNA molecules can also be used. In some instances, a DNA molecule target capture reagent can be single stranded DNA (ssDNA) or double-stranded DNA (dsDNA). In some instances, an RNA-DNA duplex is more stable than a DNA-DNA duplex and therefore provides for potentially better capture of nucleic acids.

In some instances, the disclosed methods comprise providing a selected set of nucleic acid molecules (e.g., a library catch) captured from one or more nucleic acid libraries. For example, the method may comprise: providing one or a plurality of nucleic acid libraries, each comprising a plurality of nucleic acid molecules (e.g., a plurality of target nucleic acid molecules and/or reference nucleic acid molecules) extracted from one or more samples from one or more subjects; contacting the one or a plurality of libraries (e.g., in a solution-based hybridization reaction) with one, two, three, four, five, or more than five pluralities of target capture reagents (e.g., oligonucleotide target capture reagents) to form a hybridization mixture comprising a plurality of target capture reagent/nucleic acid molecule hybrids; separating the plurality of target capture reagent/nucleic acid molecule hybrids from said hybridization mixture, e.g., by contacting said hybridization mixture with a binding entity that allows for separation of said plurality of target capture reagent/nucleic acid molecule hybrids from the hybridization mixture, thereby providing a library catch (e.g., a selected or enriched subgroup of nucleic acid molecules from the one or a plurality of libraries).

In some instances, the disclosed methods may further comprise amplifying the library catch (e.g., by performing PCR). In other instances, the library catch is not amplified.

In some instances, the target capture reagents can be part of a kit which can optionally comprise instructions, standards, buffers or enzymes or other reagents.

### Hybridization Conditions

As noted above, the methods disclosed herein may include the step of contacting the library (e.g., the nucleic acid library) with a plurality of target capture reagents to provide a selected library target nucleic acid sequences (i.e., the library catch). The contacting step can be effected in, e.g., solution-based hybridization. In some instances, the method includes repeating the hybridization step for one or more additional rounds of solution-based hybridization. In some instances, the method further includes subjecting the library catch to one or more additional rounds of solution-based hybridization with the same or a different collection of target capture reagents.

In some instances, the contacting step is effected using a solid support, e.g., an array. Suitable solid supports for hybridization are described in, e.g., Albert, T. J. et al. (2007) Nat. Methods 4(11):903-5; Hodges, E. et al. (2007) Nat. Genet. 39(12):1522-7; and Okou, D. T. et al. (2007) Nat. Methods 4(11):907-9, the contents of which are incorporated herein by reference in their entireties.

Hybridization methods that can be adapted for use in the methods herein are described in the art, e.g., as described in International Patent Application Publication No. WO 2012/092426. Methods for hybridizing target capture reagents to a plurality of target nucleic acids are described in more detail in, e.g., International Patent Application Publication No. WO 2020/236941, the entire content of which is incorporated herein by reference.

### Sequencing Methods

The methods and systems disclosed herein can be used in combination with, or as part of, a method or system for sequencing nucleic acids (e.g., a next-generation sequencing system) to generate a plurality of sequence reads that overlap one or more gene loci within a subgenomic interval in the sample and thereby determine, e.g., gene allele sequences at a plurality of gene loci. “Next-generation sequencing” (or “NGS”) as used herein may also be referred to as “massively parallel sequencing”, and refers to any sequencing method that determines the nucleotide sequence of either individual nucleic acid molecules (e.g., as in single molecule sequencing) or clonally expanded proxies for individual nucleic acid molecules in a high throughput fashion (e.g., wherein greater than 10³, 10⁴, 10⁵ or more than 10⁵ molecules are sequenced simultaneously).

Next-generation sequencing methods are known in the art, and are described in, e.g., Metzker, M. (2010) Nature Biotechnology Reviews 11:31-46, which is incorporated herein by reference. Other examples of sequencing methods suitable for use when implementing the methods and systems disclosed herein are described in, e.g., International Patent Application Publication No. WO 2012/092426. In some instances, the sequencing may comprise, for example, whole genome sequencing (WGS), whole exome sequencing, targeted sequencing, or direct sequencing. In some instances, sequencing may be performed using, e.g., Sanger sequencing.

The disclosed methods and systems may be implemented using sequencing platforms such as the Roche 454, Illumina Solexa, ABI-SOLiD, ION Torrent, Complete Genomics, Pacific Bioscience, Helicos, and/or the Polonator platform. In some instances, sequencing may comprise Illumina MiSeq sequencing. In some instances, sequencing may comprise Illumina HiSeq sequencing. In some instances, sequencing may comprise Illumina NovaSeq sequencing. Optimized methods for sequencing a large number of target genomic loci in nucleic acids extracted from a sample are described in more detail in, e.g., International Patent Application Publication No. WO 2020/236941, the entire content of which is incorporated herein by reference.

In certain instances, the disclosed methods comprise one or more of the steps of: (a) acquiring a library comprising a plurality of normal and/or tumor nucleic acid molecules from a sample; (b) simultaneously or sequentially contacting the library with one, two, three, four, five, or more than five pluralities of target capture reagents under conditions that allow hybridization of the target capture reagents to the target nucleic acid molecules, thereby providing a selected set of captured normal and/or tumor nucleic acid molecules (i.e., a library catch); (c) separating the selected subset of the nucleic acid molecules (e.g., the library catch) from the hybridization mixture, e.g., by contacting the hybridization mixture with a binding entity that allows for separation of the target capture reagent/nucleic acid molecule hybrids from the hybridization mixture, (d) sequencing the library catch to acquiring a plurality of reads (e.g., sequence reads) that overlap one or more subject intervals (e.g., one or more target sequences) from said library catch that may comprise a mutation (or alteration), e.g., a variant sequence comprising a somatic mutation or germline mutation; (e) aligning said sequence reads using an alignment method as described elsewhere herein; and/or (f) assigning a nucleotide value for a nucleotide position in the subject interval (e.g., calling a mutation using, e.g., a Bayesian method or other method described herein) from one or more sequence reads of the plurality.

In some instances, acquiring sequence reads for one or more subject intervals may comprise sequencing at least 1, at least 5, at least 10, at least 20, at least 30, at least 40, at least 50, at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, at least 1,000, at least 1,250, at least 1,500, at least 1,750, at least 2,000, at least 2,250, at least 2,500, at least 2,750, at least 3,000, at least 3,500, at least 4,000, at least 4,500, or at least 5,000 loci, e.g., genomic loci, gene loci, microsatellite loci, etc. In some instances, acquiring a sequence read for one or more subject intervals may comprise sequencing a subject interval for any number of loci within the range described in this paragraph, e.g., for at least 2,850 gene loci.

In some instances, acquiring a sequence read for one or more subject intervals comprises sequencing a subject interval with a sequencing method that provides a sequence read length (or average sequence read length) of at least 20 bases, at least 30 bases, at least 40 bases, at least 50 bases, at least 60 bases, at least 70 bases, at least 80 bases, at least 90 bases, at least 100 bases, at least 120 bases, at least 140 bases, at least 160 bases, at least 180 bases, at least 200 bases, at least 220 bases, at least 240 bases, at least 260 bases, at least 280 bases, at least 300 bases, at least 320 bases, at least 340 bases, at least 360 bases, at least 380 bases, or at least 400 bases. In some instances, acquiring a sequence read for the one or more subject intervals may comprise sequencing a subject interval with a sequencing method that provides a sequence read length (or average sequence read length) of any number of bases within the range described in this paragraph, e.g., a sequence read length (or average sequence read length) of 56 bases.

In some instances, acquiring a sequence read for one or more subject intervals may comprise sequencing with at least 100× or more coverage (or depth) on average. In some instances, acquiring a sequence read for one or more subject intervals may comprise sequencing with at least 100×, at least 150×, at least 200×, at least 250×, at least 500×, at least 750×, at least 1,000×, at least 1,500×, at least 2,000×, at least 2,500×, at least 3,000×, at least 3,500×, at least 4,000×, at least 4,500×, at least 5,000×, at least 5,500×, or at least 6,000× or more coverage (or depth) on average. In some instances, acquiring a sequence read for one or more subject intervals may comprise sequencing with an average coverage (or depth) having any value within the range of values described in this paragraph, e.g., at least 160×.

In some instances, acquiring a read for the one or more subject intervals comprises sequencing with an average sequencing depth having any value ranging from at least 100× to at least 6,000× for greater than about 90%, 92%, 94%, 95%, 96%, 97%, 98%, or 99% of the gene loci sequenced. For example, in some instances acquiring a read for the subject interval comprises sequencing with an average sequencing depth of at least 125× for at least 99% of the gene loci sequenced. As another example, in some instances acquiring a read for the subject interval comprises sequencing with an average sequencing depth of at least 4,100× for at least 95% of the gene loci sequenced.

In some instances, the relative abundance of a nucleic acid species in the library can be estimated by counting the relative number of occurrences of their cognate sequences (e.g., the number of sequence reads for a given cognate sequence) in the data generated by the sequencing experiment.

In some instances, the disclosed methods and systems provide nucleotide sequences for a set of subject intervals (e.g., gene loci), as described herein. In certain instances, the sequences are provided without using a method that includes a matched normal control (e.g., a wild-type control) and/or a matched tumor control (e.g., primary versus metastatic).

In some instances, the level of sequencing depth as used herein (e.g., an X-fold level of sequencing depth) refers to the number of reads (e.g., unique reads) obtained after detection and removal of duplicate reads (e.g., PCR duplicate reads). In other instances, duplicate reads are evaluated, e.g., to support detection of copy number alteration (CNAs).

### Alignment

Alignment is the process of matching a read with a location, e.g., a genomic location or locus. In some instances, NGS reads may be aligned to a known reference sequence (e.g., a wild-type sequence). In some instances, NGS reads may be assembled de novo. Methods of sequence alignment for NGS reads are described in, e.g., Trapnell, C. and Salzberg, S. L. Nature Biotech., 2009, 27:455-457. Examples of de novo sequence assemblies are described in, e.g., Warren R., et al., Bioinformatics, 2007, 23:500-501; Butler, J. et al., Genome Res., 2008, 18:810-820; and Zerbino, D. R. and Birney, E., Genome Res., 2008, 18:821-829. Optimization of sequence alignment is described in the art, e.g., as set out in International Patent Application Publication No. WO 2012/092426. Additional description of sequence alignment methods is provided in, e.g., International Patent Application Publication No. WO 2020/236941, the entire content of which is incorporated herein by reference.

Misalignment (e.g., the placement of base-pairs from a short read at incorrect locations in the genome), e.g., misalignment of reads due to sequence context (e.g., the presence of repetitive sequence) around an actual cancer mutation can lead to reduction in sensitivity of mutation detection, can lead to a reduction in sensitivity of mutation detection, as reads for the alternate allele may be shifted off the histogram peak of alternate allele reads. Other examples of sequence context that may cause misalignment include short-tandem repeats, interspersed repeats, low complexity regions, insertions—deletions (indels), and paralogs. If the problematic sequence context occurs where no actual mutation is present, misalignment may introduce artifactual reads of “mutated” alleles by placing reads of actual reference genome base sequences at the wrong location. Because mutation-calling algorithms for multigene analysis should be sensitive to even low-abundance mutations, sequence misalignments may increase false positive discovery rates and/or reduce specificity.

In some instances, the methods and systems disclosed herein may integrate the use of multiple, individually-tuned, alignment methods or algorithms to optimize base-calling performance in sequencing methods, particularly in methods that rely on massively parallel sequencing of a large number of diverse genetic events at a large number of diverse genomic loci. In some instances, the disclosed methods and systems may comprise the use of one or more global alignment algorithms. In some instances, the disclosed methods and systems may comprise the use of one or more local alignment algorithms. Examples of alignment algorithms that may be used include, but are not limited to, the Burrows-Wheeler Alignment (BWA) software bundle (see, e.g., Li, et al. (2009), “Fast and Accurate Short Read Alignment with Burrows-Wheeler Transform”, Bioinformatics 25:1754-60; Li, et al. (2010), Fast and Accurate Long-Read Alignment with Burrows-Wheeler Transform”, Bioinformatics epub. PMID: 20080505), the Smith-Waterman algorithm (see, e.g., Smith, et al. (1981), “Identification of Common Molecular Subsequences”, J. Molecular Biology 147(1):195-197), the Striped Smith-Waterman algorithm (see, e.g., Farrar (2007), “Striped Smith-Waterman Speeds Database Searches Six Times Over Other SIMD Implementations”, Bioinformatics 23(2):156-161), the Needleman-Wunsch algorithm (Needleman, et al. (1970) “A General Method Applicable to the Search for Similarities in the Amino Acid Sequence of Two Proteins”, J. Molecular Biology 48(3):443-53), or any combination thereof.

In some instances, the methods and systems disclosed herein may also comprise the use of a sequence assembly algorithm, e.g., the Arachne sequence assembly algorithm (see, e.g., Batzoglou, et al. (2002), “ARACHNE: A Whole-Genome Shotgun Assembler”, Genome Res. 12:177-189).

In some instances, the alignment method used to analyze sequence reads is not individually customized or tuned for detection of different variants (e.g., point mutations, insertions, deletions, and the like) at different genomic loci. In some instances, different alignment methods are used to analyze reads that are individually customized or tuned for detection of at least a subset of the different variants detected at different genomic loci. In some instances, different alignment methods are used to analyze reads that are individually customized or tuned to detect each different variant at different genomic loci. In some instances, tuning can be a function of one or more of: (i) the genetic locus (e.g., gene loci, microsatellite locus, or other subject interval) being sequenced, (ii) the tumor type associated with the sample, (iii) the variant being sequenced, or (iv) a characteristic of the sample or the subject. The selection or use of alignment conditions that are individually tuned to a number of specific subject intervals to be sequenced allows optimization of speed, sensitivity, and specificity. The method is particularly effective when the alignment of reads for a relatively large number of diverse subject intervals are optimized. In some instances, the method includes the use of an alignment method optimized for rearrangements in combination with other alignment methods optimized for subject intervals not associated with rearrangements.

In some instances, the methods disclosed herein further comprise selecting or using an alignment method for analyzing, e.g., aligning, a sequence read, wherein said alignment method is a function of, is selected responsive to, or is optimized for, one or more of: (i) tumor type, e.g., the tumor type in the sample; (ii) the location (e.g., a gene locus) of the subject interval being sequenced; (iii) the type of variant (e.g., a point mutation, insertion, deletion, substitution, copy number variation (CNV), rearrangement, or fusion) in the subject interval being sequenced; (iv) the site (e.g., nucleotide position) being analyzed; (v) the type of sample (e.g., a sample described herein); and/or (vi) adjacent sequence(s) in or near the subject interval being evaluated (e.g., according to the expected propensity thereof for misalignment of the subject interval due to, e.g., the presence of repeated sequences in or near the subject interval).

In some instances, the methods disclosed herein allow for the rapid and efficient alignment of troublesome reads, e.g., a read having a rearrangement. Thus, in some instances where a read for a subject interval comprises a nucleotide position with a rearrangement, e.g., a translocation, the method can comprise using an alignment method that is appropriately tuned and that includes: (i) selecting a rearrangement reference sequence for alignment with a read, wherein said rearrangement reference sequence aligns with a rearrangement (in some instances, the reference sequence is not identical to the genomic rearrangement); and (ii) comparing, e.g., aligning, a read with said rearrangement reference sequence.

In some instances, alternative methods may be used to align troublesome reads. These methods are particularly effective when the alignment of reads for a relatively large number of diverse subject intervals is optimized. By way of example, a method of analyzing a sample can comprise: (i) performing a comparison (e.g., an alignment comparison) of a read using a first set of parameters (e.g., using a first mapping algorithm, or by comparison with a first reference sequence), and determining if said read meets a first alignment criterion (e.g., the read can be aligned with said first reference sequence, e.g., with less than a specific number of mismatches); (ii) if said read fails to meet the first alignment criterion, performing a second alignment comparison using a second set of parameters, (e.g., using a second mapping algorithm, or by comparison with a second reference sequence); and (iii) optionally, determining if said read meets said second criterion (e.g., the read can be aligned with said second reference sequence, e.g., with less than a specific number of mismatches), wherein said second set of parameters comprises use of, e.g., said second reference sequence, which, compared with said first set of parameters, is more likely to result in an alignment with a read for a variant (e.g., a rearrangement, insertion, deletion, or translocation).

In some instances, the alignment of sequence reads in the disclosed methods may be combined with a mutation calling method as described elsewhere herein. As discussed herein, reduced sensitivity for detecting actual mutations may be addressed by evaluating the quality of alignments (manually or in an automated fashion) around expected mutation sites in the genes or genomic loci (e.g., gene loci) being analyzed. In some instances, the sites to be evaluated can be obtained from databases of the human genome (e.g., the HG19 human reference genome) or cancer mutations (e.g., COSMIC). Regions that are identified as problematic can be remedied with the use of an algorithm selected to give better performance in the relevant sequence context, e.g., by alignment optimization (or re-alignment) using slower, but more accurate alignment algorithms such as Smith-Waterman alignment. In cases where general alignment algorithms cannot remedy the problem, customized alignment approaches may be created by, e.g., adjustment of maximum difference mismatch penalty parameters for genes with a high likelihood of containing substitutions; adjusting specific mismatch penalty parameters based on specific mutation types that are common in certain tumor types (e.g. C→T in melanoma); or adjusting specific mismatch penalty parameters based on specific mutation types that are common in certain sample types (e.g. substitutions that are common in FFPE).

Reduced specificity (increased false positive rate) in the evaluated subject intervals due to misalignment can be assessed by manual or automated examination of all mutation calls in the sequencing data. Those regions found to be prone to spurious mutation calls due to misalignment can be subjected to alignment remedies as discussed above. In cases where no algorithmic remedy is found possible, “mutations” from the problem regions can be classified or screened out from the panel of targeted loci.

### Mutation Calling

Base calling refers to the raw output of a sequencing device, e.g., the determined sequence of nucleotides in an oligonucleotide molecule. Mutation calling refers to the process of selecting a nucleotide value, e.g., A, G, T, or C, for a given nucleotide position being sequenced. Typically, the sequence reads (or base calling) for a position will provide more than one value, e.g., some reads will indicate a T and some will indicate a G. Mutation calling is the process of assigning a correct nucleotide value, e.g., one of those values, to the sequence. Although it is referred to as “mutation” calling, it can be applied to assign a nucleotide value to any nucleotide position, e.g., positions corresponding to mutant alleles, wild-type alleles, alleles that have not been characterized as either mutant or wild-type, or to positions not characterized by variability.

In some instances, the disclosed methods may comprise the use of customized or tuned mutation calling algorithms or parameters thereof to optimize performance when applied to sequencing data, particularly in methods that rely on massively parallel sequencing of a large number of diverse genetic events at a large number of diverse genomic loci (e.g., gene loci, microsatellite regions, etc.) in samples, e.g., samples from a subject having cancer. Optimization of mutation calling is described in the art, e.g., as set out in International Patent Application Publication No. WO 2012/092426.

Methods for mutation calling can include one or more of the following: making independent calls based on the information at each position in the reference sequence (e.g., examining the sequence reads; examining the base calls and quality scores; calculating the probability of observed bases and quality scores given a potential genotype; and assigning genotypes (e.g., using Bayes' rule)); removing false positives (e.g., using depth thresholds to reject SNPs with read depth much lower or higher than expected; local realignment to remove false positives due to small indels); and performing linkage disequilibrium (LD)/imputation-based analysis to refine the calls.

Equations used to calculate the genotype likelihood associated with a specific genotype and position are described in, e.g., Li, H. and Durbin, R. Bioinformatics, 2010; 26(5): 589-95. The prior expectation for a particular mutation in a certain cancer type can be used when evaluating samples from that cancer type. Such likelihood can be derived from public databases of cancer mutations, e.g., Catalogue of Somatic Mutation in Cancer (COSMIC), HGMD (Human Gene Mutation Database), The SNP Consortium, Breast Cancer Mutation Data Base (BIC), and Breast Cancer Gene Database (BCGD).

Examples of LD/imputation based analysis are described in, e.g., Browning, B. L. and Yu, Z. Am. J. Hum. Genet. 2009, 85(6):847-61. Examples of low-coverage SNP calling methods are described in, e.g., Li, Y., et al., Annu. Rev. Genomics Hum. Genet. 2009, 10:387-406.

After alignment, detection of substitutions can be performed using a mutation calling method (e.g., a Bayesian mutation calling method) which is applied to each base in each of the subject intervals, e.g., exons of a gene or other locus to be evaluated, where presence of alternate alleles is observed. This method will compare the probability of observing the read data in the presence of a mutation with the probability of observing the read data in the presence of base-calling error alone. Mutations can be called if this comparison is sufficiently strongly supportive of the presence of a mutation.

An advantage of a Bayesian mutation-detection approach is that the comparison of the probability of the presence of a mutation with the probability of base-calling error alone can be weighted by a prior expectation of the presence of a mutation at the site. If some reads of an alternate allele are observed at a frequently mutated site for the given cancer type, then presence of a mutation may be confidently called even if the amount of evidence of mutation does not meet the usual thresholds. This flexibility can then be used to increase detection sensitivity for even rarer mutations/lower purity samples, or to make the test more robust to decreases in read coverage. The likelihood of a random base-pair in the genome being mutated in cancer is ˜1e-6. The likelihood of specific mutations occurring at many sites in, for example, a typical multigenic cancer genome panel can be orders of magnitude higher. These likelihoods can be derived from public databases of cancer mutations (e.g., COSMIC).

Indel calling is a process of finding bases in the sequencing data that differ from the reference sequence by insertion or deletion, typically including an associated confidence score or statistical evidence metric. Methods of indel calling can include the steps of identifying candidate indels, calculating genotype likelihood through local re-alignment, and performing LD-based genotype inference and calling. Typically, a Bayesian approach is used to obtain potential indel candidates, and then these candidates are tested together with the reference sequence in a Bayesian framework.

Algorithms to generate candidate indels are described in, e.g., McKenna, A., et al., Genome Res. 2010; 20(9):1297-303; Ye, K., et al., Bioinformatics, 2009; 25(21):2865-71; Lunter, G., and Goodson, M., Genome Res. 2011; 21(6):936-9; and Li, H., et al. (2009), Bioinformatics 25(16):2078-9.

Methods for generating indel calls and individual-level genotype likelihoods include, e.g., the Dindel algorithm (Albers, C. A., et al., Genome Res. 2011; 21(6):961-73). For example, the Bayesian EM algorithm can be used to analyze the reads, make initial indel calls, and generate genotype likelihoods for each candidate indel, followed by imputation of genotypes using, e.g., QCALL (Le S. Q. and Durbin R. Genome Res. 2011; 21(6):952-60). Parameters, such as prior expectations of observing the indel can be adjusted (e.g., increased or decreased), based on the size or location of the indels.

Methods have been developed that address limited deviations from allele frequencies of 50% or 100% for the analysis of cancer DNA. (see, e.g., SNVMix-Bioinformatics. 2010 Mar. 15; 26(6): 730-736.) Methods disclosed herein, however, allow consideration of the possibility of the presence of a mutant allele at frequencies (or allele fractions) ranging from 1% to 100% (i.e., allele fractions ranging from 0.01 to 1.0), and especially at levels lower than 50%. This approach is particularly important for the detection of mutations in, for example, low-purity FFPE samples of natural (multi-clonal) tumor DNA.

In some instances, the mutation calling method used to analyze sequence reads is not individually customized or fine-tuned for detection of different mutations at different genomic loci. In some instances, different mutation calling methods are used that are individually customized or fine-tuned for at least a subset of the different mutations detected at different genomic loci. In some instances, different mutation calling methods are used that are individually customized or fine-tuned for each different mutant detected at each different genomic loci. The customization or tuning can be based on one or more of the factors described herein, e.g., the type of cancer in a sample, the gene or locus in which the subject interval to be sequenced is located, or the variant to be sequenced. This selection or use of mutation calling methods individually customized or fine-tuned for a number of subject intervals to be sequenced allows for optimization of speed, sensitivity and specificity of mutation calling.

In some instances, a nucleotide value is assigned for a nucleotide position in each of X unique subject intervals using a unique mutation calling method, and X is at least 2, at least 3, at least 4, at least 5, at least 10, at least 15, at least 20, at least 30, at least 40, at least 50, at least 60, at least 70, at least 80, at least 90, at least 100, at least 200, at least 300, at least 400, at least 500, at least 1000, at least 1500, at least 2000, at least 2500, at least 3000, at least 3500, at least 4000, at least 4500, at least 5000, or greater. The calling methods can differ, and thereby be unique, e.g., by relying on different Bayesian prior values.

In some instances, assigning said nucleotide value is a function of a value which is or represents the prior (e.g., literature) expectation of observing a read showing a variant, e.g., a mutation, at said nucleotide position in a tumor of type.

In some instances, the method comprises assigning a nucleotide value (e.g., calling a mutation) for at least 10, 20, 40, 50, 60, 70, 80, 90, 100, 200, 300, 400, 500, 600, 700, 800, 900, or 1,000 nucleotide positions, wherein each assignment is a function of a unique value (as opposed to the value for the other assignments) which is or represents the prior (e.g., literature) expectation of observing a read showing a variant, e.g., a mutation, at said nucleotide position in a tumor of type.

In some instances, assigning said nucleotide value is a function of a set of values which represent the probabilities of observing a read showing said variant at said nucleotide position if the variant is present in the sample at a specified frequency (e.g., 1%, 5%, 10%, etc.) and/or if the variant is absent (e.g., observed in the reads due to base-calling error alone).

In some instances, the mutation calling methods described herein can include the following: (a) acquiring, for a nucleotide position in each of said X subject intervals: (i) a first value which is or represents the prior (e.g., literature) expectation of observing a read showing a variant, e.g., a mutation, at said nucleotide position in a tumor of type X; and (ii) a second set of values which represent the probabilities of observing a read showing said variant at said nucleotide position if the variant is present in the sample at a frequency (e.g., 1%, 5%, 10%, etc.) and/or if the variant is absent (e.g., observed in the reads due to base-calling error alone); and (b) responsive to said values, assigning a nucleotide value (e.g., calling a mutation) from said reads for each of said nucleotide positions by weighing, e.g., by a Bayesian method described herein, the comparison among the values in the second set using the first value (e.g., computing the posterior probability of the presence of a mutation), thereby analyzing said sample.

Additional description of mutation calling methods is provided in, e.g., International Patent Application Publication No. WO 2020/236941, the entire content of which is incorporated herein by reference.

### Systems for CNA Detection and Calling

Also disclosed herein are systems designed to implement any of the disclosed methods. The systems may comprise, e.g., one or more processors, and a memory unit communicatively coupled to the one or more processors and configured to store instructions that, when executed by the one or more processors, cause the system to: receive, at the one or more processors, sequence read data for a plurality of sequence reads associated with a plurality of nucleic acid molecules obtained from a sample from a subject; generate for a plurality of loci, using the one or more processors, a minor allele coverage ratio and a major allele coverage ratio; segment, using the one or more processors, a genome into a plurality of genomic segments (for example, based on the minor allele coverage ratio, the major allele coverage ratio, or a total coverage ratio); generate, using the one or more processors, for loci in the plurality of loci, copy number grid model input data comprising (i) a difference between the major allele coverage ratio and the minor allele coverage ratio, and (ii) the sum of the major allele coverage ratio and the minor allele coverage ratio; fit, using the one or more processors, a plurality of copy number grid models comprising allowed copy number states to the copy number model input data; select, using the one or more processors, a copy number grid model from the plurality of copy number grid models; and assign, using the one or more processors, a copy number state for at least a portion of the plurality of genomic segments based on the selected copy number grid model. The different copy number grid models in the plurality of copy number grid models may be initialized using different initial tumor purity estimates and tumor ploidy estimates.

In some implementations, the instructions that cause the system to select the selected copy number grid model comprise instructions that cause the system to: determine, for each genomic segment, a distribution of the copy number grid model input data; identify, for each genomic segment, a distance between the distribution and a closest copy number state; and determine an overall model fit score based on an average distance across the plurality of genomic segments.

In some implementations, the instructions that fit allowed copy number states to the copy number grid model input data comprise instructions that cause the system to: fit the allowed copy numbers states to the copy number grid model input data based on an initial tumor purity estimate and an initial tumor ploidy estimate; and iteratively: (a) assign a preliminary copy number to each segment in the plurality of segments; (b) determine an updated tumor ploidy estimate and updated tumor purity estimate based on the preliminary copy number assignments; and (c) re-fit the allowed copy numbers states to the copy number grid model input data based on the updated tumor ploidy estimate and updated tumor purity estimate.

In some implementations, the system further comprises an electronic display. The instructions may further comprise instructions that, when executed by the one or more processors, cause the system to overlay the selected copy number grid model with the transformed coverage ratio data, and present the overlay using an interface of the electronic display.

In some implementations, the system further comprises instructions that, when executed by the one or more processors, cause the system to call a copy number alteration for the one or more genetic loci or one or more genomic segments based on one or more assigned copy number states or a sum coverage ratio threshold.

In some implementations, the system further comprises instructions that, when executed by the one or more processors, cause the system to generate a report indicating the copy number state or called copy number alteration for one or more genomic loci or one or more genomic segments. The instructions may further cause the system to transmit the report to the subject or a healthcare provider, for example via a computer network or a peer-to-peer connection.

In some instances, the disclosed systems may further comprise a sequencer, e.g., a next generation sequencer (also referred to as a massively parallel sequencer). Examples of next generation (or massively parallel) sequencing platforms include, but are not limited to, the Roche 454, Illumina Solexa, ABI-SOLiD, ION Torrent, or Pacific Bioscience sequencing platforms.

In some instances, the disclosed systems may be used for calling of CNAs in any of a variety of samples as described herein (e.g., a tissue sample, biopsy sample, hematological sample, or liquid biopsy sample derived from the subject).

In some instances, the plurality of gene loci for which sequencing data is processed to determine copy number alterations may comprise at least 1, 2, 3, 4, 5, 6, 7, 8, 9, 10, 20, 30, 40, 50, 60, 70, 80, 90, 100, or more than 10 gene loci.

In some instance, the nucleic acid sequence data is acquired using a next generation sequencing technique (also referred to as a massively parallel sequencing technique) having a read-length of less than 400 bases, less than 300 bases, less than 200 bases, less than 150 bases, less than 100 bases, less than 90 bases, less than 80 bases, less than 70 bases, less than 60 bases, less than 50 bases, less than 40 bases, or less than 30 bases.

In some instances, the determination of copy number alterations in one or more gene loci is used to select, initiate, adjust, or terminate a treatment for cancer in the subject (e.g., a patient) from which the sample was derived, as described elsewhere herein.

In some instances, the disclosed systems may further comprise sample processing and library preparation workstations, microplate-handling robotics, fluid dispensing systems, temperature control modules, environmental control chambers, additional data storage modules, data communication modules (e.g., Bluetooth®, WiFi, intranet, or internet communication hardware and associated software), display modules, one or more local and/or cloud-based software packages (e.g., instrument/system control software packages, sequencing data analysis software packages), etc., or any combination thereof. In some instances, the systems may comprise, or be part of, a computer system or computer network as described elsewhere herein.

A non-transitory computer-readable storage medium, which may be part of the system described herein or independent of such a system, may store one or more programs that include instructions, which when executed by one or more processors of a system, cause the system to: receive, at the one or more processors, sequence read data for a plurality of sequence reads associated with a plurality of nucleic acid molecules obtained from a sample from a subject; generate for a plurality of loci, using the one or more processors, a minor allele coverage ratio and a major allele coverage ratio; segment, using the one or more processors, a genome into a plurality of genomic segments (for example, based on the minor allele coverage ratio, the major allele coverage ratio, or a total coverage ratio); generate, using the one or more processors, for loci in the plurality of loci, copy number grid model input data that includes (i) a difference between the major allele coverage ratio and the minor allele coverage ratio, and (ii) the sum of the major allele coverage ratio and the minor allele coverage ratio; fit, using the one or more processors, a plurality of copy number grid models comprising allowed copy number states to the transformed coverage ratio data, copy number grid model input data; select, using the one or more processors, a selected copy number grid model from the plurality of copy number grid models; and assign, using the one or more processors, a copy number state for at least a portion of the plurality of genomic segments based on the selected copy number grid model. The different copy number grid models in the plurality of copy number grid models may be initialized using different initial tumor purity estimates and tumor ploidy estimates.

In some implementations of the non-transitory computer-readable storage medium, the instructions that cause the system to select the selected copy number grid model comprise instructions that cause the system to: determine, for each genomic segment, a distribution of the copy number grid model input data; identify, for each genomic segment, a distance between the distribution and a closest copy number state; and determine an overall model fit score based on an average distance across the plurality of genomic segments.

In some implementations of the non-transitory computer-readable storage medium, the instructions that cause the system to fit allowed copy number states to the copy number grid model input data comprise instructions that cause the system to: fit the allowed copy numbers states to the copy number grid model input data based on an initial tumor purity estimate and an initial tumor ploidy estimate; and iteratively: (a) assign a preliminary copy number to each segment in the plurality of segments; (b) determine an updated tumor ploidy estimate and updated tumor purity estimate based on the preliminary copy number assignments; and (c) re-fit the allowed copy numbers states to the copy number grid model input data based on the updated tumor ploidy estimate and updated tumor purity estimate.

In some implementations of the non-transitory computer-readable storage medium, the instructions further comprise instructions that, when executed by the one or more processors, cause the system to overlay the selected copy number grid model with the copy number grid model input data, and present the overlay using an interface of an electronic display.

In some implementations, the non-transitory computer-readable storage medium further comprises instructions that, when executed by the one or more processors, cause the system to call a copy number alteration for the one or more genetic loci or one or more genomic segments based on one or more assigned copy number states or a sum coverage ratio threshold.

In some implementations, the non-transitory computer-readable storage medium further comprises instructions that, when executed by the one or more processors, cause the system to generate a report indicating the copy number state or called copy number alteration for one or more genomic loci or one or more genomic segments. The insurrections may further cause the system to transmit the report to the subject or a healthcare provider, for example via a computer network or a peer-to-peer connection.

### Computer Systems and Networks

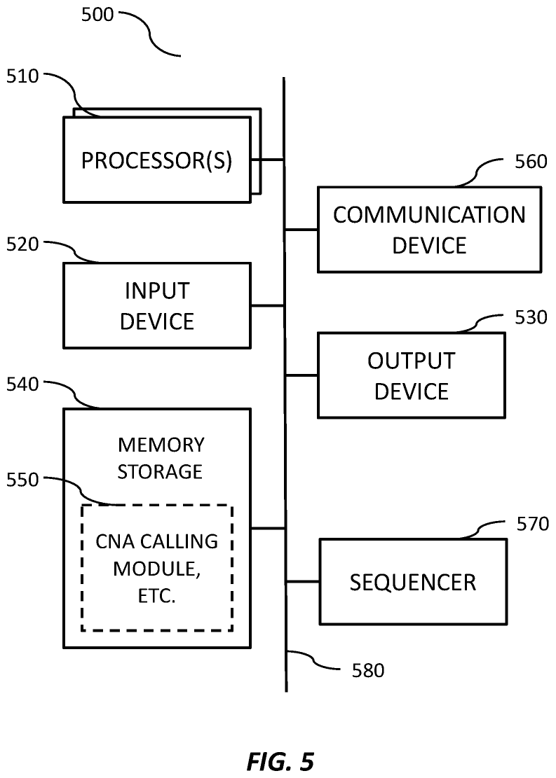

FIG. 5 illustrates an example of a computing device or system in accordance with one embodiment. Device 500 can be a host computer connected to a network. Device 500 can be a client computer or a server. As shown in FIG. 5, device 500 can be any suitable type of microprocessor-based device, such as a personal computer, workstation, server or handheld computing device (portable electronic device) such as a phone or tablet. The device can include, for example, one or more processor(s) 510, input devices 520, output devices 530, memory or storage devices 540, communication devices 560, and nucleic acid sequencers 570. Software 550 residing in memory or storage device 540 may comprise, e.g., an operating system as well as software for executing the methods described herein. Input device 520 and output device 530 can generally correspond to those described herein, and can be either connectable or integrated with the computer.

Input device 520 can be any suitable device that provides input, such as a touch screen, keyboard or keypad, mouse, or voice-recognition device. Output device 530 can be any suitable device that provides output, such as a touch screen, haptics device, or speaker. The input device 520 and the output device 530 can be the same device or different devices.

Storage 540 can be any suitable device that provides storage (e.g., an electrical, magnetic or optical memory including a RAM (volatile and non-volatile), cache, hard drive, or removable storage disk). Communication device 560 can include any suitable device capable of transmitting and receiving signals over a network, such as a network interface chip or device. The components of the computer can be connected in any suitable manner, such as via a wired media (e.g., a physical system bus 580, Ethernet connection, or any other wire transfer technology) or wirelessly (e.g., Bluetooth®, Wi-Fi®, or any other wireless technology).

Software module 550, which can be stored as executable instructions in storage 540 and executed by processor(s) 510, can include, for example, an operating system and/or the processes that embody the functionality of the methods of the present disclosure (e.g., as embodied in the devices as described herein).

Software module 550 can also be stored and/or transported within any non-transitory computer-readable storage medium for use by or in connection with an instruction execution system, apparatus, or device, such as those described herein, that can fetch instructions associated with the software from the instruction execution system, apparatus, or device and execute the instructions. In the context of this disclosure, a computer-readable storage medium can be any medium, such as storage 540, that can contain or store processes for use by or in connection with an instruction execution system, apparatus, or device. Examples of computer-readable storage media may include memory units like hard drives, flash drives and distribute modules that operate as a single functional unit. Also, various processes described herein may be embodied as modules configured to operate in accordance with the embodiments and techniques described above. Further, while processes may be shown and/or described separately, those skilled in the art will appreciate that the above processes may be routines or modules within other processes.

Software module 550 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

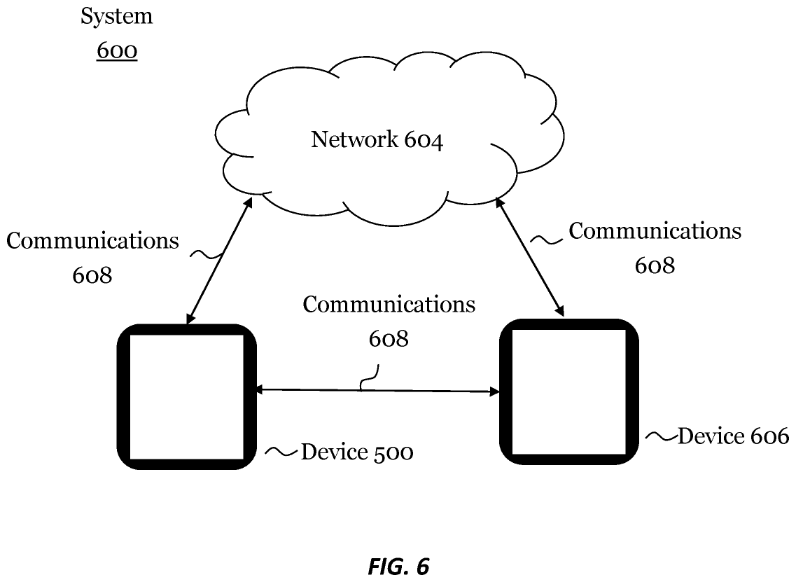

Device 500 may be connected to a network (e.g., network 604, as shown in FIG. 6 and/or described below), which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 500 can be implemented using any operating system, e.g., an operating system suitable for operating on the network. Software module 550 can be written in any suitable programming language, such as C, C++, Java or Python. In various embodiments, application software embodying the functionality of the present disclosure can be deployed in different configurations, such as in a client/server arrangement or through a Web browser as a Web-based application or Web service, for example. In some embodiments, the operating system is executed by one or more processors, e.g., processor(s) 510.

Device 500 can further include a sequencer 570, which can be any suitable nucleic acid sequencing instrument.

FIG. 6 illustrates an example of a computing system in accordance with one embodiment. In system 600, device 500 (e.g., as described above and illustrated in FIG. 5) is connected to network 604, which is also connected to device 606. In some embodiments, device 606 is a sequencer. Exemplary sequencers can include, without limitation, Roche/454's Genome Sequencer (GS) FLX System, Illumina/Solexa's Genome Analyzer (GA), Illumina's HiSeq 2500, HiSeq 3000, HiSeq 4000 and NovaSeq 6000 Sequencing Systems, Life/APG's Support Oligonucleotide Ligation Detection (SOLiD) system, Polonator's G.007 system, Helicos BioSciences' HeliScope Gene Sequencing system, or Pacific Biosciences' PacBio RS system.

Devices 500 and 606 may communicate, e.g., using suitable communication interfaces via network 604, such as a Local Area Network (LAN), Virtual Private Network (VPN), or the Internet. In some embodiments, network 604 can be, for example, the Internet, an intranet, a virtual private network, a cloud network, a wired network, or a wireless network. Devices 500 and 606 may communicate, in part or in whole, via wireless or hardwired communications, such as Ethernet, IEEE 802.11b wireless, or the like. Additionally, devices 500 and 606 may communicate, e.g., using suitable communication interfaces, via a second network, such as a mobile/cellular network. Communication between devices 500 and 606 may further include or communicate with various servers such as a mail server, mobile server, media server, telephone server, and the like. In some embodiments, Devices 500 and 706 can communicate directly (instead of, or in addition to, communicating via network 604), e.g., via wireless or hardwired communications, such as Ethernet, IEEE 802.11b wireless, or the like. In some embodiments, devices 500 and 606 communicate via communications 608, which can be a direct connection or can occur via a network (e.g., network 604).

One or all of devices 500 and 606 generally include logic (e.g., http web server logic) or are programmed to format data, accessed from local or remote databases or other sources of data and content, for providing and/or receiving information via network 604 according to various examples described herein.

It should be understood from the foregoing that, while particular implementations of the disclosed methods and systems have been illustrated and described, various modifications can be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents.

1. A method comprising:
providing a plurality of nucleic acid molecules obtained from a sample from a subject;
ligating one or more adapters onto one or more nucleic acid molecules from the plurality of nucleic acid molecules;
amplifying the one or more ligated nucleic acid molecules from the plurality of nucleic acid molecules;
capturing amplified nucleic acid molecules from the amplified nucleic acid molecules;
sequencing, by a sequencer, the captured nucleic acid molecules to obtain a plurality of sequence reads that represent the captured nucleic acid molecules, thereby generating sequence read data for a genome of the sample;
receiving, at one or more processors, the sequence read data;
generating for a plurality of genetic loci, using the one or more processors, a minor allele coverage ratio and a major allele coverage ratio;
segmenting, using the one or more processors, the genome into a plurality of genomic segments;
generating, using the one or more processors, for genetic loci in the plurality of genetic loci, copy number grid model input data comprising (i) a difference between the major allele coverage ratio and the minor allele coverage ratio, and (ii) a sum of the major allele coverage ratio and the minor allele coverage ratio;
fitting, using the one or more processors, a plurality of copy number grid models comprising allowed copy number states to the copy number grid model input data;
selecting, using the one or more processors, a copy number grid model from the plurality of copy number grid models; and
assigning, using the one or more processors, a copy number state for at least a portion of the plurality of genomic segments based on the selected copy number grid model.

2-8. (canceled)

9. A method, comprising:
receiving, at one or more processors, sequence read data for a plurality of sequence reads associated with a plurality of nucleic acid molecules obtained from a sample from a subject;
generating for a plurality of genetic loci, using the one or more processors, a minor allele coverage ratio and a major allele coverage ratio;
segmenting, using the one or more processors, a genome into a plurality of genomic segments;
generating, using the one or more processors, for genetic loci in the plurality of genetic loci, copy number grid model input data comprising (i) a difference between the major allele coverage ratio and the minor allele coverage ratio, and (ii) a sum of the major allele coverage ratio and the minor allele coverage ratio;
fitting, using the one or more processors, a plurality of copy number grid models comprising allowed copy number states to the copy number grid model input data;
selecting, using the one or more processors, a copy number grid model from the plurality of copy number grid models; and
assigning, using the one or more processors, a copy number state for at least a portion of the plurality of genomic segments based on the selected copy number grid model

10. The method of claim 9, wherein the segmenting is based on the minor allele coverage ratio, the major allele coverage ratio, or a total coverage ratio.

11. The method of claim 9, wherein selecting the copy number grid model from the plurality of copy number grid models comprises:
determining, for each genomic segment, a distribution of the copy number grid model input data;
identifying, for each genomic segment, a distance between the distribution and a closest copy number state; and
determining an overall model fit score based on an average distance across the plurality of genomic segments.

12. The method of claim 9, wherein different copy number grid models in the plurality of copy number grid models are initialized using different initial tumor purity estimates and tumor ploidy estimates.

13. The method of claim 9, wherein fitting the plurality of copy number grid models to the copy number grid model input data comprises, for each copy number grid model:
fitting the allowed copy numbers states of the copy number grid model to copy number grid model input data based on an initial tumor purity estimate and an initial tumor ploidy estimates; and
iteratively:
assigning a preliminary copy number to each genomic segment in the plurality of genomic segments;
determining an updated tumor ploidy estimate and updated tumor purity estimate based on the preliminary copy number assignments; and
re-fitting the allowed copy numbers states to the copy number grid model input data based on the updated tumor ploidy estimate and updated tumor purity estimate.

14. The method of claim 12, wherein the initial tumor purity is bound by a preselected tumor purity lower limit and a preselected tumor purity upper limit.

15. (canceled)

16. The method of claim 12, wherein the initial tumor ploidy is bound by a preselected tumor ploidy lower limit or a tumor ploidy upper limit.

17-19. (canceled)

20. The method of claim 9, wherein the assigned copy number state for each of the plurality of genomic segments is a total copy number count for the genomic segment, a minor allele copy number count for the genomic segment, or a major allele copy number count for the genomic segment.

21-22. (canceled)

23. The method of claim 9, further comprising overlaying the selected copy number grid model and the copy number grid model input data to generate an overlay; and displaying the overlay.

24. (canceled)

25. The method of claim 9, further comprising calling a copy number alteration for the one or more genetic loci or one or more genomic segments based on one or more assigned copy number states or a sum coverage ratio threshold.

26. The method of claim 25, wherein the called copy number alteration for the one or more genetic loci or the one or more genomic segments is used to diagnose or confirm a diagnosis of disease in the subject.

27. The method of claim 25, further comprising generating a genomic profile for the subject comprising the called copy number alteration for the one or more genetic loci or the one or more genomic segments.

28-40. (canceled)

41. A method of selecting an anticancer therapy for a subject having cancer, the method comprising:
responsive to a copy number alteration for the one or more genetic loci or the one or more genomic segments called according to the method of claim 25, selecting an anticancer therapy for the subject.

42. The method of claim 41, further comprising determining an effective amount of an anticancer therapy to administer to the subject based on the called copy number alteration for the one or more genetic loci or the one or more genomic segments.

43. The method of claim 42, further comprising administering the anticancer therapy to the subject based on the called copy number alteration for the one or more genetic loci or the one or more genomic segments.

44. A method of treating a cancer in a subject, comprising:
responsive to a called copy number alteration for the one or more genetic loci or the one or more genomic segments called according to the method of claim 25, administering an effective amount of an anticancer therapy to the subject.

45. A method for monitoring tumor progression or recurrence in a subject, the method comprising:
calling a copy number alteration for one or more genetic loci or one or more genomic segments using a first sample obtained from the subject at a first time point according to the method of claim 25;
calling a copy number alteration for the one or more genetic loci or one or more genomic segments using a second sample obtained from the subject at a second time point; and
comparing the first called a copy number alteration to the second called a copy number alteration for the one or more genetic loci or the one or more genomic segments, thereby monitoring the cancer progression or recurrence.

46-55. (canceled)

56. The method of claim 9, further comprising generating, by the one or more processors, a report indicating the copy number state or called copy number alteration for one or more genomic loci or one or more genomic segments.

57-67. (canceled)

68. A system comprising:
one or more processors; and
a memory communicatively coupled to the one or more processors and configured to store instructions that, when executed by the one or more processors, cause the system to:
receive, at the one or more processors, sequence read data for a plurality of sequence reads associated with a plurality of nucleic acid molecules obtained from a sample from a subject;
generate for a plurality of loci, using the one or more processors, a minor allele coverage ratio and a major allele coverage ratio;
segment, using the one or more processors, a genome into a plurality of genomic segments;
generate, using the one or more processors, for loci in the plurality of loci, copy number grid model input data comprising (i) a difference between the major allele coverage ratio and the minor allele coverage ratio, and (ii) a sum of the major allele coverage ratio and the minor allele coverage ratio;
fit, using the one or more processors, a plurality of copy number grid models comprising allowed copy number states to the copy number grid model input data;
select, using the one or more processors, a copy number grid model from the plurality of copy number grid models; and
assign, using the one or more processors, a copy number state for at least a portion of the plurality of genomic segments based on the selected copy number grid model.

69-105. (canceled)
